SEQUENCE LISTING (13 sequences)

SEQ ID NO: 1 - DNA, 32 bp, synthetic construct
  misc_feature 1..32: note = F-Primer Left Homology Arm
  source 1..32: mol_type = other DNA
tcgagctcggtacccgggcaggcgaggcgccg

SEQ ID NO: 2 - DNA, 24 bp, synthetic construct
  misc_feature 1..24: note = R-Primer Left Homology Arm
  source 1..24: mol_type = other DNA
ggatgcccggcgccagtccgcgtt

SEQ ID NO: 3 - DNA, 29 bp, synthetic construct
  misc_feature 1..29: note = F-Primer Right Homology Arm
  source 1..29: mol_type = other DNA
gactggcgccgggcatccttgatggaaag

SEQ ID NO: 4 - DNA, 38 bp, synthetic construct
  misc_feature 1..38: note = F-Primer Right Homology Arm
  source 1..38: mol_type = other DNA
tgcaggtcgactctagagcgttcgtggatttcaagggc

SEQ ID NO: 5 - DNA, 500 bp, synthetic construct
  misc_feature 1..500: note = Left Homology Arm (500 bp)
  source 1..500: mol_type = other DNA
caggcgaggcgccgcgtgtatttgattgataccaacgtcatcagcgaaacgcgcaagcgcgagcgcgccaaccccggcgtgcgcgcgttcttccggcaggcggcgcgggaaggtgccgcgctctacctgtcggcgctgaccgtgggcgagctccagcgtggcgtagcgctgatccgccatcgcggcgatacggcgcaggccgagctgctggagcaatggctggcgaccgtgctggaggattttggccggctggtgttgccggtcgatgccgacgtcgcccaggtctggggccagctgcgcgcgccgcggcctgagcacgcgctggacaagttcattgccgccaccgcgctgatccatgacctgaccattgtcacgcgcaatgttgaggatttccgcggcacgggcgcgatgctgctgaatccgttcacctagccccacctcaaaagaaaggaccccgcatagcggggccctgctcacgtcagcggaacgcggactggcgc

SEQ ID NO: 6 - DNA, 500 bp, synthetic construct
  misc_feature 1..500: note = Right Homology Arm (500 bp)
  source 1..500: mol_type = other DNA
cgggcatccttgatggaaagcaggacataaaggacttgagccacatggccggcatgaggttccccgctgcggcgacatgtggctctgacgtcaggtgtggcggccattctcggggccagccttccggcaaacgccggcattgtaatgcgctcaggtcttcggcagtgtgacaccgtgctggccctggtacttgccgccgcggtcgcggtacgaggtctcgcagacttcgtcgctctcgaagaacagcacctgggcgcaaccctcgccggcgtagatcttggcgggcagcggcgtggtgttggagaactccagcgtcacatagccttcccactccggctcgaacggcgtcacattgacgatgatgccgcagcgggcgtaggtgctcttgcccaggcagatggtcagcacgctgcgcgggatccggaagtattccatcgtgcgcgccagcgcgaacgaattgggcgggatgatgcagacatcgcccttgaaatccacgaacg

SEQ ID NO: 7 - DNA, 5719 bp, synthetic construct
  misc_feature 1..5719: note = pK18mobsacB empty suicide plasmid
  source 1..5719: mol_type = other DNA
tgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgataagctagcttcacgc

SEQ ID NO: 8 - protein, 350 aa, Pseudomonas syringae
MTNLQTFELPTEVTGCAADISLGRALIQAWQKDGIFQIKTDSEQDRKTQEAMAASKQFCKEPLTFKSSCVSDLTYSGYVASGEEVTAGKPDFPEIFTVCKDLSVGDQRVKAGWPCHGPVPWPNNTYQKSMKTFMEELGLAGERLLKLTALGFELPINTFTDLTRDGWHHMRVLRFPPQTSTLSRGIGAHTDYGLLVIAAQDDVGGLYIRPPVEGEKRNRNWLPGESSAGMFEHDEPWTFVTPTPGVWTVFPGDILQFMTGGQLLSTPHKVKLNTRERFACAYFHEPNFEASAYPLFEPSANERIHYGEHFTNMFMRCYPDRITTQRINKENRLAHLEDLKKYSDTRATGS

SEQ ID NO: 9 - protein, 347 aa, Ralstonia solanacearum
MTGLTTFHLPERILHSEAHRQLGQDMVAAWRADGIFQIALSTPQQHTTDEAFAQSRRFFELDFETKRRHVSELTYSGYIASREEITAGEADYSEIFTICPDIGMDDVRVREGWPCHGPVPWPGTAYRDRMTDFTGMLGAFGERLLQLTALGLGLDDMETFTRLTRDGWHHMRVLRFPTVQSSENARGIGAHTDYGLLVIAAQDDVGGLYVRPPIAGERRNRNWLPSESTAGMFEHDDGWTFIKPEPAVLTVFPGDFLQFLTGGHLMSTPHKVRLNTRERFAMAYFHEPNFDAWVEPLKADADTDVAPIHYGTHFTNMFMRCYPKRITTRRIEEQGLLDRLPALGEVA

SEQ ID NO: 10 - protein, 369 aa, Microcoleus sp.
  source 1..369: note = Microcoleus asticus
MTHKYQEKIEVSNLQIFHLPESITGIQSDIDIARQMIQAWRRDGIFHVAVNKIQERKSERTFAASRRFFGMPLESKSQFISDLTYSGYIASGEEVTAGESDYSEIFTVCKDVPLNDRRVQAQWPCHGPAPWPDEDYQQSMKAYMDELGSIGEKLLKLTALGLELDDINALTELTKDGWHHMRVLRFPALSQKSTRGIGAHTDYGLLVIAAQDDVGGLYIRPPVEGEKRNRNWLPTESMGGMYENEEPWILVKPVPSVLTVFPGDILQFLTNGYLLSTPHKVRLNTRERFAIAYFHEPNFEACVRPLFAPSSDEHIHYGSHFTNMFMRCYPDRITTRRIIDENRLSILGVLKNEGLRRLTTAKKAIELQR

SEQ ID NO: 11 - protein, 360 aa, Myxococcus stipitatus
MIELETFQLPQSVSGREADIALGLTMVRAWRRDGIFQVRMSPAQAEKSQRAFELSRHFFRQSLETKARCVSDLTYSGYIASGQELTASEADLSEVFTVCRDVPLTDPRVQSKWPCHGPGPWPDESWRQGMQAHAEELGSVGERLLRLIALGLGLDIDALTTLTHDGWHHMRVLRFPARSPTTTRGIGAHTDYGLLVIAAQDDVGGLYVRPPVEGEKRPRNWLPHESSAGMYEHDEPWTYVKPVPGVLTVFPGDILQFLTRGYLLSTPHKVVLNTRERFALAYFHEPQFEACVRPLSAPTRDEYIHYGTHFTNMFMRSYPDRVTTQRILDESRLTTLSWLRQEAVLRTAPLEAVPLQRAAG

SEQ ID NO: 12 - protein, 361 aa, Scytonema sp.
  source 1..361: note = Nostoc sp. ATCC 43529
MTDLQTFDLPKSITGSQSDIDLAHQMIQAWRTDGIFQVATNAIQTRKTENAFEASKRFFRMPLDFKSQCISNLTYSGYIASGEEITAGESDYSEIFTICKDVRLDDVRVQAQWPCHGSVPWPDNNYHQNMKAFMDELGIMGEKLLKLVALGLELDDIDALTKLTRDGWHHMRVLRFPALSEKSTRGIGAHTDYGLLVIAAQDDVGGLYVRPPVEGEKRNRNWLSDESSAGMYENDQPWTFVKPVPKVLTVFPGDILQFMTHNYLLSTPHKVRLNTRERFALAYFHEPNFQACVRPLFDSSNDDYIHYGTHFTNMFMRCYPYRITTRRILDEDRLSVLELLRNEALGGMLRPKYKTLVPSYL

SEQ ID NO: 13 - protein, 359 aa, Scytonema sp.
  source 1..359: note = Scytonema sp. NIES-4073
MTDLQTFHLPKSITGTQSDIDTAREIIQAWRTDGIFQVATNTIQDRKTESAFEASRRFFRMPMKFKSQCISDLNYGGYIASGEEVTAGKSDYSEIYTICKDIPLNDARVQAQWPCHGPMPWPDQEYHQSMKVFMDELGLIGEKLLKLTALGLGLDDINALTKLTRDGWHHMRVLRFPTLSQKSARGIGAHTDYGLLVIAAQDDVGGLYIRPPVEGEKRNRNWLSTESMAGMYENDDPWTFVKPVPSVLTVFPGDILQFLTNGYLLSTPHKVRLNTRERFALAYFHEPNFDACVRPLFDPSSDEHIHYGTHFTNMFMRCYADRITTRRIINEDRLSILARLENKTLGRLTTMKNAYALQR

(19) United States
(12) Patent Application Publication
(10) Pub. No.: US 2023/0407271 A1
(43) Pub. Date: Dec. 21, 2023

(54) MICROORGANISMS AND METHODS FOR THE CONTINUOUS PRODUCTION OF ETHYLENE FROM C1-SUBSTRATES

(71) Applicant: LanzaTech, Inc., Skokie, IL (US)

(72) Inventors: Sean Dennis Simpson, Evanston, IL (US); Jennifer Rosa Holmgren, Skokie, IL (US); James MacAllister Clomburg, Chicago, IL (US); Jim Jeffrey Daleiden, Niles, IL (US); Audrey Jean Harris, Chicago, IL (US); Stephanie Rhianon Jones, Evanston, IL (US); Michael Koepke, Chicago, IL (US); Timothy James Politano, Chicago, IL (US)

(21) Appl. No.: 18/339,050

(22) Filed: Jun. 21, 2023

Related U.S. Application Data
(60) Provisional application No. 63/366,758, filed on Jun. 21, 2022.
(60) Provisional application No. 63/506,350, filed on Jun. 5, 2023.
(60) Provisional application No. 63/506,351, filed on Jun. 5, 2023.

(51) Int. Cl. C12N 9/02; C12P 5/02

(52) U.S. Cl. CPC C12N 9/0069; C12Y 113/12019; C12P 5/026

(57) ABSTRACT

Methods and microorganisms are genetically engineered to continuously produce ethylene by microbial fermentation, particularly by the microbial fermentation of a gaseous substrate. The microorganisms are C1-fixing. Further, the gaseous substrate comprises CO₂ and an energy source. The production of ethylene can be improved by varying promoters or nutrient limiting means.

### CROSS REFERENCE TO RELATED APPLICATIONS

This application claims the benefit of U.S. Provisional Patent Application Nos. 63/366,758 filed on Jun. 21, 2022, 63/506,350 filed on Jun. 5, 2023, and 63/506,351 filed on Jun. 5, 2023, and the entirety of these U.S. Provisional Patent Applications are incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on May 26, 2023, is named LT245US1-Sequences.xml and is 24,485 bytes in size.

### FIELD

The present disclosure relates to genetically engineered microorganisms and methods for the continuous production of ethylene by microbial fermentation, particularly by microbial fermentation of a gaseous substrate.

### BACKGROUND

It has long been recognized that catalytic processes, such as the Fischer-Tropsch process, may be used to convert gases containing carbon dioxide (CO₂), carbon monoxide (CO), and/or hydrogen (H₂), such as industrial waste gas or syngas, into a variety of fuels and chemicals. Recently, however, gas fermentation has emerged as an alternative platform for the biological fixation of such gases. In particular, C1-fixing microorganisms have been demonstrated to convert gases containing CO₂, CO, and/or H₂ into products such as ethanol and 2,3-butanediol. Ethylene is the most widely produced organic compound in the world, useful in a broad spectrum of industries including plastics, solvents, and textiles. Ethylene is currently produced by steam cracking fossil fuels or dehydrogenating ethane. With millions of metric tons of ethylene being produced each year, however, more than enough carbon dioxide is produced by such processes to greatly contribute to the global carbon footprint. The production of ethylene through renewable methods would accordingly help to meet the huge demand from the energy and chemical industries, while also helping to protect the environment. Efficient production of such chemical products may be limited, however, by slow microbial growth, limited gas uptake, sensitivity to toxins, or diversion of carbon substrates into undesired by-products. There is accordingly an ongoing and unmet need to develop an efficient production of ethylene by microbial fermentation of a gaseous substrate that can be produced easily from renewable resources, and which would offer a broad array of useful applications.

### SUMMARY

It is against the above background that the present disclosure provides certain advantages and advancements over the prior art.

Although this disclosure disclosed herein is not limited to specific advantages or functionalities, the disclosure provides a method and a genetically engineered microorganism capable of producing ethylene from a gaseous substrate, the microorganism comprising a heterologous nucleic acid encoding an ethylene-forming enzyme (EFE).

In some aspects of the method disclosed herein, the microorganism is a recombinant C1-fixing microorganism capable of producing ethylene from a gaseous substrate comprising a nucleic acid encoding a group of exogenous enzymes comprising ethylene-forming enzyme (EFE).

In some aspects of the microorganism disclosed herein, the microorganism is directed to a recombinant C1-fixing microorganism capable of switching cellular burden in production of ethylene, the microorganism comprising a nucleic acid encoding a group of exogenous enzymes comprising ethylene-forming enzyme (EFE) and one or more inducible promoters.

The microorganism of an embodiment, further comprising a nucleic acid encoding a group of exogenous enzymes comprising alpha-ketoglutarate permease (AKGP), wherein the nucleic acid is operably linked to a promoter.

The microorganism of an embodiment, wherein the microorganism is selected from the group consisting of Cupriavidus necator and Ralstonia eutropha.

The microorganism of an embodiment, wherein the microorganism is Cupriavidus necator.

The microorganism of an embodiment, further comprising a nucleic acid encoding alpha-ketoglutarate, wherein the nucleic acid is codon optimized for expression in the microorganism.

The microorganism of an embodiment, wherein the one or more inducible promoters is selected from an H₂ inducible promoter, a phosphate limited inducible promoter, a nitrogen limited inducible promoter, a CO₂ inducible promoter, or any combination thereof.

The microorganism of an embodiment, wherein the CO₂ inducible promoter is CBB.

The microorganism of an embodiment, wherein the EFE is codon optimized for expression in the microorganism.

The microorganism of an embodiment, further comprising a disruptive mutation in one or more genes.

The microorganism of an embodiment, wherein ethylene is converted into a derivative material selected from polyethylene (PE), polyethylene terephthalate (PET), polyvinyl chloride (PVC), ethylene vinyl acetate (EVA), sustainable aviation fuel (SAF), or any combination thereof.

The microorganism of an embodiment, wherein the gaseous substrate comprises CO₂ and an energy source.

The microorganism of an embodiment, wherein the gaseous substrate comprises CO₂, and H₂, O₂, or both.

One embodiment is directed to a method for the continuous production of ethylene, the process comprising: passing a gaseous substrate to a bioreactor containing a culture of a recombinant C1-fixing microorganism according to claim 1, in a culture medium such that the microorganism converts the gaseous substrate to ethylene; and recovering the ethylene from the bioreactor.

One embodiment is directed to a method of culturing the microorganism according to claim 1, comprising growing the microorganism in a medium comprising a gaseous substrate, wherein the gaseous substrate comprises CO₂.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the energy source is H₂.

One embodiment is a directed to a method comprising growing the microorganism in a medium comprising a gaseous substrate, wherein the gaseous substrate comprises CO₂ and an energy source.

The method of an embodiment further comprises co-producing ethylene and microbial biomass.

The method of an embodiment, wherein switching the cellular burden comprises a step of limiting the intracellular oxygen concentration.

The method of an embodiment, wherein the microbial biomass is suitable as animal feed.

The method of an embodiment, wherein the gaseous substrate further comprises H₂, O₂, or both.

In some aspects of the microorganism disclosed herein, the microorganism produces a commodity chemical product, microbial biomass, single cell protein (SCP), one or more intermediates, or any combination thereof.

In some aspects of the microorganism disclosed herein, the microorganism is derived from a parental bacterium selected from the group consisting of Cupriavidus necator.

In some aspects of the microorganism disclosed herein, where the product is selected from the group 1-butanol, butyrate, butene, butadiene, methyl ethyl ketone, ethylene, acetone, isopropanol, lipids, 3-hydroxypropionate, terpenes, isoprene, fatty acids, fatty alcohols, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, or monoethylene glycol.

The disclosure further provides the genetically engineered C1-fixing microorganism, further comprising a microbial biomass and at least one excipient.

The disclosure further provides the genetically engineered C1-fixing microorganism, wherein the animal feed is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The disclosure further provides the genetically engineered C1-fixing microorganism, wherein the microorganism is suitable as a single cell protein (SCP).

The disclosure further provides the genetically engineered C1-fixing microorganism, wherein the microorganism is suitable as a cell-free protein synthesis (CFPS) platform.

The disclosure further provides the genetically engineered C1-fixing microorganism, wherein the product is native to the microorganism.

In some aspects of the method disclosed herein, the substrate comprises one or more of CO, CO₂, and H₂.

In some embodiments, both anaerobic and aerobic gases can be used to feed separate cultures (e.g., an anaerobic culture and an aerobic culture) in two or more different bioreactors that are both integrated into the same process stream.

In some embodiments, the disclosure provides a method for storing energy in the form of a biopolymer comprising intermittently processing at least a portion of electric energy generated from a renewable and/or non-renewable energy source in an electrolysis process to produce at least H₂, O₂ or CO; intermittently passing at least one of H₂, O₂, or CO from the electrolysis process to a bioreactor containing a culture comprising a liquid nutrient medium and a microorganism capable of producing a biopolymer; and fermenting the culture.

In an embodiment, the disclosure also provides a system for storing energy in the form of biopolymer comprising an electrolysis process in intermittent fluid communication with a renewable and/or non-renewable energy source for producing at least one of H₂, O₂, or CO; an industrial plant for producing at least C1 feedstock; a bioreactor, in intermittent fluid communication with the electrolysis process and/or in continuous fluid communication with the industrial plant, comprising a reaction vessel suitable for intermittently growing, fermenting, and/or culturing and housing a microorganism capable of producing a biopolymer.

In some embodiments, the disclosure provides a method for improving the performance and/or the economics of a fermentation process, the fermentation process defining a bioreactor containing a bacterial culture in a liquid nutrient medium, wherein the method comprises passing a C1 feedstock comprising one or both of CO and CO₂ from an industrial process to the bioreactor, wherein the C1 feedstock has a cost per unit, intermittently passing at least one of H₂, O₂, or CO from the electrolysis process to the bioreactor, wherein the electrolysis process has a cost per unit, and fermenting the culture to produce one or more fermentation products, wherein each of the one or more fermentation products has a value per unit. In certain instances, multiple electrolysis processes are utilized in order to provide one or all of CO, CO₂, and H₂ to the bioreactor.

In another embodiment, the local power grid provides electricity intermittently passed as electrical energy produced by power based on availability of electrical power or the availability of electricity below a threshold price, where power prices fall as demand falls, or as set by the local power grid.

In an embodiment, the disclosure can be operated intermittently by storing energy in the form of a biopolymer, where product conversion can be intermittent during periods when an electricity grid is oversupplied with electricity, or idle when electricity is scarce or power is in demand. The disclosure provides a process that is capable of being fine-tuned to assist with balancing an electrical power grid system by storing energy in the form of a biopolymer.

In one embodiment an autotrophic microorganism intermittently consumes, in part or entirely, the energy provided by the availability of power.

In one embodiment, the systems disclosed herein relate to generating fine bubbles and may include a vessel containing a liquid, a plate comprising a plurality of orifices positioned in an upper portion of the vessel and configured to accelerate at least a portion of the liquid in the vessel, and at least one sparger positioned within the vessel with a surface of the sparger positioned from about 50 mm to about 300 mm, 500 mm, or 1000 mm from a bottom of the plate. The sparger may be configured to inject bubbles into the liquid. In some examples, the sparger may be positioned within the vessel to create a first zone for the bubbles to rise within the vessel, and to create a second zone for the accelerated liquid to break the bubbles into fine bubbles and for fluid to flow through the vessel. The fluid may include the accelerated portion of the liquid and fine bubbles. In still other examples, the superficial velocity of the gas phase in the vessel may be at least 30 mm/s. The sparger may be a sintered sparger or an orifice sparger. The thickness of the plate may be about 1 mm to about 25 mm. The accelerated liquid may have a velocity of about 8000 mm/s to about 17000 mm/s. In other examples, the accelerated liquid may have a velocity of about 12000 mm/s to about 17000 mm/s. In some examples, the bubbles injected into the liquid from the sparger may have a diameter of about 2 mm to about 20 mm. In another example, the bubbles injected into the liquid from the sparger may have a diameter of about 5 mm to about 15 mm, or from about 7 mm to about 13 mm. The fine bubbles may have a diameter of about 0.1 mm to about 5 mm, or about 0.2 mm to about 1.5 mm. The plurality of orifices may also be configured to accelerate at least 90% of the liquid in the vessel.

In another embodiment, the methods disclosed herein relate to generating fine bubbles that may include sparging gas into a vessel containing a liquid via at least one sparger positioned within the vessel and configured to inject bubbles into the liquid and accelerating a portion of the liquid in the vessel via a perforated plate positioned in an upper portion of the vessel, in which the liquid may be accelerated from the plate to break the bubbles into fine bubbles. In some examples, a superficial velocity of the gas phase in the vessel may be at least 30 mm/s. In other examples, the superficial velocity of the gas phase in the vessel may be from about 30 mm/s to about 80 mm/s. The sparger may be a sintered sparger or an orifice sparger. The liquid may be accelerated from the perforated plate at a velocity of about 8000 mm/s to about 17000 mm/s. In some examples, the liquid may be accelerated from the perforated plate at a velocity of about 12000 mm/s to about 17000 mm/s. The bubbles injected into the liquid from the sparger may have a diameter of about 2 mm to about 20 mm, or from greater than 5 mm to about 15 mm, or from about 7 mm to about 13 mm. Often the bubbles injected into the liquid from the sparger are not spherical. The injected bubbles may be referred to as coarse bubbles. In contrast, the fine bubbles may have a diameter of about 0.1 mm to about 5 mm, or about 0.2 mm to about 1.5 mm. The fine bubbles are typically spherical. The liquid stream may be introduced at a location proximate to the plate. The sparger may be positioned perpendicular or parallel to the plate, and a top or side surface of the sparger may be positioned from about 50 mm to about 300 mm, 500 mm, or 1000 mm from a bottom of the plate.

In yet another embodiment, the systems disclosed herein relate to a bioreactor that may include a vessel containing a liquid growth medium, a plate that may include a plurality of orifices positioned in an upper portion of the vessel and configured to accelerate at least a portion of the liquid growth medium in the vessel, a substrate that may include at least one C1 carbon source, at least one sparger positioned within the vessel with a surface of the sparger that may be positioned from about 50 mm to about 300 mm, 500 mm, or 1000 mm from a bottom of the plate and the sparger configured to inject substrate bubbles into the liquid growth medium. The sparger positioned within the vessel may create a first zone for the substrate bubbles to rise within the vessel, and a second zone for the accelerated liquid growth medium to break the substrate bubbles into substrate fine bubbles, and for fluid to flow through the vessel. The fluid may have the accelerated portion of the liquid growth medium and may have the substrate fine bubbles, and a culture of at least one microorganism in the liquid growth medium. The culture of at least one microorganism may anaerobically ferment the substrate to produce at least one fermentation product.

In still another embodiment, the methods disclosed herein relate to generating substrate fine bubbles in a bioreactor and may include sparging substrate bubbles of at least one C1 carbon source into a vessel containing a liquid growth medium via at least one sparger positioned within the vessel and accelerating a portion of the liquid growth medium in the vessel via a perforated plate positioned in an upper portion of the vessel. The liquid growth medium accelerated from the plate may break the substrate bubbles into substrate fine bubbles. A superficial velocity of the gas phase in the vessel may be at least 30 mm/s. A culture of at least one microorganism may be included in the liquid growth medium and may anaerobically ferment the substrate to produce at least one fermentation product.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

These and other aspects of the present disclosure, which should be considered in all its novel aspects, will become apparent from the following description, which is given by way of example only, with reference to the accompanying figures, in which:

FIG. 1 shows a schematic showing pathways for CO2 fixation, central carbon metabolism, and the TCA cycle in Cupriavidus necator with heterologous expression of ethylene forming enzyme for ethylene production.

FIG. 2 shows ethylene production by Cupriavidus necator strains with ethylene forming enzyme expression (pBBR1-Efe) and the blank vector control (pBBR1) when grown on formate as the sole carbon and energy source.

FIG. 3 shows continuous ethylene production from CO₂ as the sole carbon source in a CSTR over an 11-day period by a Cupriavidus necator strain with ethylene forming enzyme expression (pBBR1-Efe).

FIG. 4 shows a schematic flux balance analysis predicted gene knockout strategies (red arrows) to eliminate unwanted by-products during ethylene production from CO₂ and H₂ in Cupriavidus necator. Gene annotations are provided below each enzyme name (See Table 1 for additional details).

FIG. 5 schematically depicts a system for generating bubbles within a vessel, according to the systems and methods disclosed herein.

FIG. 6 shows ethylene production by Cupriavidus necator strains with ethylene forming enzyme (Efe) expressed via a constitutive or phosphate-limited inducible promoter along with the blank vector control (pBBR1) when grown in phosphate limited minimal media.

FIG. 7 shows ethylene production by Cupriavidus necator strains with ethylene forming enzyme variants from various organisms expressed via a chemically inducible promoter (rhamnose). Accession numbers for EFE variant: Pseudomonas syringae (AAD16440.1), Microcoleus asticus (NQE34890), Myxococcus stipitatus (WP_015351455.1), Nostoc sp. ATCC 43529 (RCJ18531), Ralstonia solanacearum (WP_014618742.1), Scytonema sp. NIES-4073 (WP_096562523.1).

FIG. 8 shows continuous ethylene production from CO2 as the sole carbon source in a CSTR over a 5.5-day period by a Cupriavidus necator strain with ethylene forming enzyme expressed via a phosphate-limited inducible promoter. CSTR was operated under phosphate-limited conditions starting at day ˜14.7.

FIG. 9 shows continuous ethylene production from CO2 as the sole carbon source in a CSTR over a 14-day period by a Cupriavidus necator strain with ethylene forming enzyme expressed via synthetic CbbL promoter.

FIG. 10 shows ethylene production from CO2 as the sole carbon source during CSTR start-up by a Cupriavidus necator strain with ethylene forming enzyme expressed via synthetic soluble hydrogenase promoter.

FIG. 11 shows increasing FeSO₄×7H₂O concentration results in increased ethylene production in cells grown on fructose under PO₄-limited conditions.

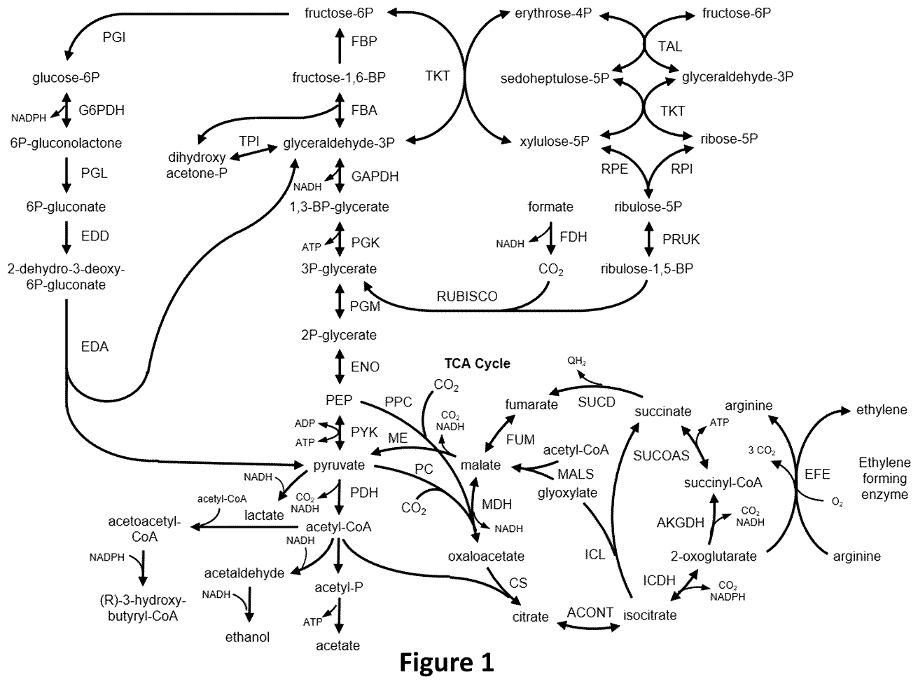

### DETAILED DESCRIPTION

The following description of embodiments is given in general terms. The disclosure is further elucidated from the disclosure given under the heading “Examples” herein below, which provides experimental data supporting the disclosure, specific examples of various aspects of the disclosure, and means of performing the disclosure.

The inventors have surprisingly been able to engineer a C1-fixing microorganism to continuously produce ethylene.

Unless otherwise defined, the following terms as used throughout this specification are defined as follows:

The disclosure provides microorganisms for the biological co-production of proteins, chemicals, and microbial biomass. A “microorganism” is a microscopic organism, especially a bacterium, archaeon, virus, or fungus. In an embodiment, the microorganism of the disclosure is a bacterium.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility. The microorganisms of the disclosure are non-naturally occurring.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization. The microorganisms of the disclosure are genetically engineered.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms. The microorganisms of the disclosure are generally recombinant.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure.

Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

In particular, a heterologous nucleic acid or protein expressed in the microorganism described herein may be derived from Bacillus, Clostridium, Cupriavidus, Escherichia, Gluconobacter, Hyphomicrobium, Lysinibacillus, Paenibacillus, Pseudomonas, Sedimenticola, Sporosarcina, Streptomyces, Thermithiobacillus, Thermotoga, Zea, Klebsiella, Mycobacterium, Salmonella, Mycobacteroides, Staphylococcus, Burkholderia, Listeria, Acinetobacter, Shigella, Neisseria, Bordetella, Streptococcus, Enterobacter, Vibrio, Legionella, Xanthomonas, Serratia, Cronobacter, Cupriavidus, Helicobacter, Yersinia, Cutibacterium, Francisella, Pectobacterium, Arcobacter, Lactobacillus, Shewanella, Erwinia, Sulfurospirillum, Peptococcaceae, Thermococcus, Saccharomyces, Pyrococcus, Glycine, Homo, Ralstonia, Brevibacterium, Methylobacterium, Geobacillus, bos, gallus, Anaerococcus, Xenopus, Amblyrhynchus, rattus, mus, sus, Rhodococcus, Rhizobium, Megasphaera, Mesorhizobium, Peptococcus, Agrobacterium, Campylobacter, Acetobacterium, Alkalibaculum, Blautia, Butyribacterium, Eubacterium, Moorella, Oxobacter, Sporomusa, Thermoanaerobacter, Schizosaccharomyces, Paenibacillus, Fictibacillus, Lysinibacillus, Ornithinibacillus, Halobacillus, Kurthia, Lentibacillus, Anoxybacillus, Solibacillus, Virgibacillus, Alicyclobacillus, Sporosarcina, Salimicrobium, Sporosarcina, Planococcus, Corynebacterium, Thermaerobacter, Sulfobacillus, or Symbiobacterium.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof.

Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

The term “copolymer” is a composition comprising two or more species of monomers are linked in the same polymer chain of the disclosure.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

“Disrupted gene” refers to a gene that has been modified in some way to reduce or eliminate expression of the gene, regulatory activity of the gene, or activity of an encoded protein or enzyme. The disruption may partially inactivate, fully inactivate, or delete the gene or enzyme. The disruption may be a knockout (KO) mutation that fully eliminates the expression or activity of a gene, protein, or enzyme. The disruption may also be a knock-down that reduces, but does not entirely eliminate, the expression or activity of a gene, protein, or enzyme. The disruption may be anything that reduces, prevents, or blocks the biosynthesis of a product produced by an enzyme. The disruption may include, for example, a mutation in a gene encoding a protein or enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, RNAi, TALEN, siRNA, CRISPR, or CRISPRi) or protein which inhibits the expression of a protein or enzyme. The disruption may be introduced using any method known in the art. For the purposes of the present disclosure, disruptions are laboratory-generated, not naturally occurring.

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism.

The microorganism of the disclosure may be derived from essentially any parental microorganism.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes.

The microorganism of the disclosure may be further classified based on functional characteristics. For example, the microorganism of the disclosure may be or may be derived from a C1-fixing microorganism, an aerobe, an anaerobe, an acetogen, an ethanologen, a carboxydotroph, an autotroph, and/or a methanotroph. The microorganism of the disclosure may be selected from chemoautotroph, hydrogenotroph, knallgas, methanotroph, or any combination thereof. In some embodiments, the microorganism may be hydrogen-oxidizing, carbon monoxide-oxidizing, knallgas, or any combination thereof, with the capability to grow and synthesize biomass on gaseous carbon sources such as syngas and/or CO₂, such that the production microorganisms synthesize targeted chemical products under gas cultivation. The microorganisms and methods of the present disclosure can enable low cost synthesis of biochemicals, which can compete on price with petrochemicals and higher-plant derived amino acids, proteins, and other biological nutrients. In certain embodiments, these amino acids, proteins, and other biological nutrients may have a substantially lower price than amino acids, proteins, and other biological nutrients produced through heterotrophic or microbial phototrophic synthesis. Knallgas microbes, hydrogenotrophs, carboxydotrophs, and chemoautotrophs more broadly, are able to capture CO₂ or CO as their sole carbon source to support biological growth. In some embodiments, this growth includes the biosynthesis of amino acids and proteins. Knallgas microbes and other hydrogenotrophs can use H₂ as a source of reducing electrons for respiration and biochemical synthesis. In some embodiments of the present invention knallgas organisms and/or hydrogenotrophs and/or carboxydotrophs and/or other chemoautotrophic microorganisms are grown on a stream of gasses including but not limited to one or more of the following: CO₂; CO; H₂; along with inorganic minerals dissolved in aqueous solution. In some embodiments knallgas microbes and/or hydrogenotrophs and/or carboxydotrophs and/or other chemoautotrophic and/or methanotrophic microorganisms convert greenhouse gases into biomolecules including amino acids and proteins.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source. Often, the microorganism of the disclosure is a C1-fixing bacterium. In a preferred embodiment, the microorganism of the disclosure is derived from a C1-fixing microorganism.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen), sometimes referred to as “microoxic conditions.” Often, the microorganism of the disclosure is an anaerobe. In a preferred embodiment, the microorganism of the disclosure is derived from an anaerobe.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, NY, 2006). All naturally occurring acetogens are C1-fixing, anaerobic, autotrophic, and non-methanotrophic. Often, the microorganism of the disclosure is an acetogen. In a preferred embodiment, the microorganism of the disclosure is derived from an acetogen.

An “ethanologen” is a microorganism that produces or is capable of producing ethanol. Often, the microorganism of the disclosure is an ethanologen. In a preferred embodiment, the microorganism of the disclosure is derived from an ethanologen.

An “autotroph” is a microorganism capable of growing in the absence of organic carbon. Instead, autotrophs use inorganic carbon sources, such as CO and/or CO₂. Often, the microorganism of the disclosure is an autotroph. In a preferred embodiment, the microorganism of the disclosure is derived from an autotroph.

A “carboxydotroph” is a microorganism capable of utilizing CO as a sole source of carbon and energy. Often, the microorganism of the disclosure is a carboxydotroph. In a preferred embodiment, the microorganism of the disclosure is derived from a carboxydotroph.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

The term “knallgas” refers to the mixture of molecular hydrogen and oxygen gas. A “knallgas microorganism” is a microbe that can use hydrogen as an electron donor and oxygen as an electron acceptor in respiration for the generation of intracellular energy carriers such as Adenosine-5′-triphosphate (ATP).

The terms “oxyhydrogen” and “oxyhydrogen microorganism” can be used synonymously with “knallgas” and “knallgas microorganism” respectively. Knallgas microorganisms generally use molecular hydrogen by means of hydrogenases, with some of the electrons donated from H₂ being utilized for the reduction of NAD⁺ (and/or other intracellular reducing equivalents) and some of the electrons from H₂ being used for aerobic respiration. Knallgas microorganisms generally fix CO₂ autotrophically, through pathways including but not limited to the Calvin Cycle or the reverse citric acid cycle.

As described above, however, the microorganism of the disclosure may also be derived from essentially any parental microorganism, such as a parental microorganism selected from the group consisting of Escherichia coli and Saccharomyces cerevisiae.

In another embodiment, the microorganism of the disclosure is an aerobic bacterium. In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the invention include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the microorganism is Cupriavidus necator DSM248 or DSM541.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In the microorganisms of the disclosure, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes. In an embodiment, limitation selected from nutrients, dissolved oxygen, or any combination thereof diverts carbon flux to desired products.

In an embodiment, the fermentation broth comprises the feed streams in combination with the aerobic microorganism in the bioreactor. In some embodiments, the feed streams, e.g., a carbon source feed stream, a flammable gas-containing stream, and an oxygen-containing gas feed stream, react with the microorganism in the bioreactor to at least partially form the fermentation broth (which may also include other products, byproducts, and other media fed to the bioreactor). The unreacted oxygen, or the oxygen that is not consumed by the microorganism, exists as both dissolved oxygen and gaseous oxygen in a dispersed gaseous phase within the fermentation broth. The same holds true for the other gases that are soluble. The dispersed gaseous phase, containing the unreacted components, e.g., oxygen, nitrogen, hydrogen, carbon dioxide and/or water vapor, rises to the headspace of the bioreactor.

In some embodiments, an oxygen-containing gas, e.g., air, can be fed directly into the fermentation broth. In one embodiment, the oxygen-containing gas can be an oxygen-enriched source, e.g., oxygen-enriched air or pure oxygen. In an embodiment, the oxygen-containing gas may comprise greater than 6.0 vol. % of oxygen, e.g., greater than 10.0 vol. %, greater than 20.0 vol. %, greater than 40.0 vol. %, greater than 60.0 vol. %, greater than 80.0 vol. %, or greater than 90.0 vol. %. In some embodiments, the oxygen-containing gas may be pure oxygen.

In one embodiment, the microorganism of the disclosure is capable of producing ethylene. One embodiment is directed to a recombinant C1-fixing microorganism capable of producing ethylene from a carbon source comprising a nucleic acid encoding a group of exogenous enzymes comprising at least one ethylene forming enzyme (EFE). In some embodiments the EFE is derived from Pseudomonas syringae. In an embodiment, the EFE has an E.C. number 1.13.12.19. The microorganism of an embodiment comprising at least one EFE having an E.C. number 1.13.12.19. The microorganism of an embodiment, further comprising a nucleic acid encoding a group of exogenous enzymes comprising at least one alpha-ketoglutarate permease (AKGP).

The microorganism of an embodiment, wherein a nucleic acid encoding a group of exogenous enzymes comprises at least one EFE, at least one AKGP, or any combination thereof. The microorganism of an embodiment, wherein a nucleic acid encoding a group of exogenous enzymes comprises at least one EFE and at least one AKGP. The microorganism of an embodiment, wherein the nucleotide encoding a group of exogenous enzymes is inserted into a bacterial vector plasmid, a high copy number bacterial vector plasmid, a bacterial vector plasmid having an inducible promoter, a nucleotide guide of a homologous recombination system, a CRISPR Cas system, or any combination thereof. In an embodiment, the promoter is a phosphate limited inducible promoter. In some embodiments, the promoter is a nitrogen limited promoter. In some embodiments, the promoter is an NtrC-P activated promoter. In some embodiments, the promoter is a H₂ inducible promoter. In one embodiment, the microorganism comprises an intracellular oxygen concentration limit. In another embodiment, the method limits intracellular oxygen concentration. In one embodiment, the method comprises a step of controlling dissolved oxygen. In an embodiment, the method comprises decreased ethylene production with decreased dissolved oxygen concentration. In some embodiments, the microorganism comprises a molecular switch. In some embodiments, the microorganism comprises an ability to switch the cellular burden under variable conditions.

In some embodiments, the microorganism is a natural or an engineered microorganism that is capable of converting a gaseous substrate as a carbon and/or energy source. In one embodiment, the gaseous substrate includes CO₂ as a carbon source. In some embodiments, the gaseous substrate includes H₂, and/or O₂ as an energy source. In one embodiment, the gaseous substrate includes a mixture of gases, comprising H₂ and/or CO₂ and/or CO.

In some embodiments, the gas fermentation product is selected from an alcohol, an acid, a diacid, an alkene, a terpene, an isoprene, and alkyne. In some embodiments, the method and microorganism disclosed herein are for the improved production of ethylene. In an embodiment, the method and microorganism disclosed herein are for the improved production of a gas fermentation product.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1propanol, 1hexanol, 1octanol, chorismate-derived products, 3hydroxybutyrate, 1,3butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monoethylene glycol, or any combination thereof.

The disclosure provides microorganisms capable of producing ethylene comprising culturing the microorganism of the disclosure in the presence of a substrate, whereby the microorganism produces ethylene.

The enzymes of the disclosure may be codon optimized for expression in the microorganism of the disclosure. “Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the disclosure are codon optimized for expression in the microorganism of the disclosure. Although codon optimization refers to the underlying genetic sequence, codon optimization often results in improved translation and, thus, improved enzyme expression. Accordingly, the enzymes of the disclosure may also be described as being codon optimized.

One or more of the enzymes of the disclosure may be overexpressed. “Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The enzymes of the disclosure may comprise a disruptive mutation. A “disruptive mutation” refers to a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

Although exemplary sequences and sources for enzymes are provided herein, the disclosure is by no means limited to these sequences and sources—it also encompasses variants. The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like.

Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. The promoter may be a Wood-Ljungdahl pathway promoter, a ferredoxin promoter, a pyruvate ferredoxin oxidoreductase promoter, an Rnf complex operon promoter, an ATP synthase operon promoter, or a phosphotransacetylase/acetate kinase operon promoter.

It should be appreciated that the disclosure may be practiced using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants.” By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein.

The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practiced using polypeptides whose sequence varies from the amino acid sequences specifically exemplified herein. These variants may be referred to herein as “functionally equivalent variants.” A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps: introduction into a shuttle microorganism of (i) of an expression construct/vector as described herein and (ii) a methylation construct/vector comprising a methyltransferase gene; expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thiogalactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage ΦT1 methyltransferase and the methyltransferase described in the Examples herein after may be used. Nucleic acids encoding suitable methyltransferases will be readily appreciated having regard to the sequence of the desired methyltransferase and the genetic code.

Any number of constructs/vectors adapted to allow expression of a methyltransferase gene may be used to generate the methylation construct/vector.

In one embodiment, the substrate comprises CO₂ and an energy source. In some embodiments, the substrate comprises CO₂ and an energy source. In an embodiment, the substrate comprises CO₂, H₂, and O₂. In some embodiments, the substrate comprises CO₂ and any suitable energy source. In one embodiment, the substrate comprises CO. In one embodiment, the substrate comprises CO2 and CO. In another embodiment, the substrate comprises CO2 and H2. In another embodiment, the substrate comprises CO2 and CO and H2.

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H₂, N₂, or electrons.

In some embodiments, the gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

In addition to ethylene, the microorganism of the disclosure may be cultured to produce one or more co-products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to ethylene. In certain embodiments, microbial biomass itself may be considered a product. These products may be further converted to produce at least one component of diesel, jet fuel, sustainable aviation fuel (SAF) and/or gasoline. In certain embodiments, ethylene may be catalytically converted into another product, article, or any combination thereof. Additionally, the microbial biomass may be further processed to produce a single cell protein (SCP) by any method or combination of methods known in the art. In addition to one or more target chemical products, the microorganism of the disclosure may also produce ethanol, acetate, and/or 2,3-butanediol. In another embodiment, the microorganism and methods of the disclosure improve the production of products, proteins, microbial biomass, or any combination thereof.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived. Ethylene is not known to be produced by any naturally-occurring microorganism, such that it is a non-native product of all microorganisms.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product, such as ethylene glycol, accounts for at least about 5%, 10%, 15%, 20%, 30%, 50%, or 75% of all fermentation products produced by the microorganism of the disclosure. In one embodiment, ethylene accounts for at least 10% of all fermentation products produced by the microorganism of the disclosure, such that the microorganism of the disclosure has a selectivity for ethylene glycol of at least 10%. In another embodiment, ethylene accounts for at least 30% of all fermentation products produced by the microorganism of the disclosure, such that the microorganism of the disclosure has a selectivity for ethylene of at least 30%.

At least one of the one or more fermentation products may be biomass produced by the culture. At least a portion of the microbial biomass may be converted to a single cell protein (SCP). At least a portion of the single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO2, and H2.

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavour, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

Herein, reference to an acid (e.g., acetic acid or 2-hydroxyisobutyric acid) should be taken to also include the corresponding salt (e.g., acetate or 2-hydroxyisobutyrate).

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of ethylene glycol. If necessary, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

A “sparger” may comprise a device to introduce gas into a liquid, injected as bubbles, to agitate it or to dissolve the gas in the liquid. Example spargers may include orifice spargers, sintered spargers, and drilled pipe spargers. In certain configurations drilled pipe spargers may be mounted horizontally. In other examples, spargers may be mounted vertically or horizontally. In some examples, the sparger may be a perforated plate or ring, sintered glass, sintered steel, porous rubber pipe, porous metal pipe, porous ceramic or stainless steel, drilled pipe, stainless steel drilled pipe, polymeric drilled pipe, etc. The sparger may be of various grades (porosities) or may include certain sized orifices to produce a specific sized bubble or range of bubble sizes.

A “vessel”, “reaction vessel”, or “column” may be a vessel or container in which one or more gas and liquid streams, or flows may be introduced for bubble generation and/or fine bubble generation, and for subsequent gas-liquid contacting, gas-absorption, biological or chemical reaction, or surface-active material adsorption. In a reaction vessel, the gas and liquid phases may flow in the vertical directions. In a reaction vessel, larger bubbles from a sparger, having a buoyancy force larger than the drag force imparted by the liquid, may rise upwards. Smaller fine bubbles, having a buoyancy force less than or equal to the drag force imparted by the liquid, may flow downward with the liquid, as described by the systems and methods disclosed herein. A column or reaction vessel may not be restricted to any specific aspect (height to diameter) ratio. A column or reaction vessel may also not be restricted to any specific material and can be constructed from any material suitable to the process such as stainless steel, PVC, carbon steel, or polymeric material. A column or reaction vessel may contain internal components such as one or more static mixers that are common in biological and chemical engineering processing. A reaction vessel may also consist of external or internal heating or cooling elements such as water jackets, heat exchangers, or cooling coils. The reaction vessel may also be in fluid contact with one or more pumps to circulate liquid, bubbles, fine bubbles, and or one or more fluids of the system.

A “perforated plate” or “plate” may comprise a plate or similar arrangement designed to facilitate the introduction of liquid or additional liquid into the vessel that may be in the form of multiple liquid jets (i.e., accelerated liquid flow). The perforated plate may have a plurality of pores or orifices evenly or unevenly distributed across the plate that allow the flow of liquid from a top of the plate to the bottom of the plate. In some examples, the orifices may be spherical-shaped, rectangular-shaped, hexagonal prism-shaped, conical-shaped, pentagonal prism-shaped, cylindrical-shaped, frustoconical-shaped, or round-shaped. In other examples, the plate may comprise one or more nozzles adapted to generate liquid jets which flow into the column. The plate may also contain channels in any distribution or alignment where such channels are adapted to receive liquid and facilitate flow through into the reaction vessel. The plate may be made of stainless steel with a predefined number of laser-burnt, machined, or drilled pores or orifices. The specific orifice size may depend upon the required fine bubble size and required liquid, fine bubble, and/or fluid velocities. A specific orifice shape may be required to achieve the proper liquid acceleration and velocity from the plate to break or shear the sparger bubbles into the desired fine bubble size, and to create enough overall fluid downflow to carry the fine bubbles and liquid downward in the reaction vessel. The shape of the orifice may also impact ease of manufacturing and related costs. According to one embodiment, a straight orifice may be optimal due to ease of manufacture.

The systems and methods as disclosed herein, employ, within a vessel, multiple liquid jets or portions of accelerated liquid flow generated using the perforated plate to accelerate liquid and break bubbles into smaller fine bubbles having a greater superficial surface area than the original bubbles. The original bubbles are initially generated by injecting gas with a sparger positioned entirely within the reaction vessel. In one example, original bubbles injected into liquid from a sparger may have a diameter of about 2 mm to about 20 mm. In another example, original bubbles injected into liquid from a sparger may have a diameter of about 5 mm to about 15 mm. In other examples, original bubbles injected into liquid from a sparger may have a diameter of about 7 mm to about 13 mm. Upon injection, the original bubbles subsequently migrate upwards through the liquid and encounter the multiple liquid jets or portions of accelerated liquid flow which breaks the original bubbles into fine bubbles. The resulting fine bubbles and liquid flow down the reactor vessel in the downward fluid flow. The fine bubbles of substrate provide a carbon source and optionally an energy source to the microbes which then produce one or more desired products. The spargers are positioned within the vessel to create a first zone for the original bubbles to rise within the vessel, and to create a second zone for the accelerated liquid to break the original bubbles into fine bubbles and for fluid to flow through the vessel, where the fluid comprises the accelerated portion of the liquid and fine bubbles.

Due to the nature of the multi-phase system, one approach to maximizing product generation is to increase gas to liquid mass transfer. The more gas substrate transferred to a reaction liquid, the greater the desired product generated. The smaller fine bubbles of the present disclosure provide an increased superficial surface area resulting in an increased gas to liquid mass transfer rates overcoming known solubility issues. Additionally, the downflow reactor systems disclosed herein are effective to increase the residence time of the fine bubbles. The increased time that the fine bubbles remain in the reaction liquid generally provides increased amounts of reaction product generated, as well as greater surface areas in contact with the microbes. As such, the systems and methods disclosed herein improve over previous systems by generating fine bubbles that maximize gas to liquid superficial surface areas leading to high gas to liquid mass transfer rates. Further, the systems and methods disclosed herein provide superficial gas and liquid velocities not achieved by the previous systems and methods resulting in the generation of fine bubbles with high gas phase residence time resulting in the efficient creation of chemical and biological reaction products.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor. Purification techniques may include affinity tag purification (e.g. His, Twin-Strep, and FLAG), bead-based systems, a tip-based approach, and FPLC system for larger scale, automated purifications. Purification methods that do not rely on affinity tags (e.g. salting out, ion exchange, and size exclusion) are also disclosed.

In some embodiments, the produced chemical product may be isolated and enriched, including purified, using any suitable separation and/or purification technique known in the art. In an embodiment, the produced chemical product is gaseous. In one embodiment, the chemical product is a liquid. In an embodiment, a gaseous chemical product may pass a filter, a gas separation membrane, a gas purifier, or any combination thereof. In one embodiment, the chemical product is separated by an absorbent column. In another embodiment, the chemical product is stored in one or more cylinders after separation. In one embodiment, the chemical product is integrated into an infrastructure or process of an oil, gas, refinery, petrochemical operation, or any combination thereof. The infrastructure or process may be existing or new. In an embodiment, the gas fermentation product is integrated into oil and gas production, transportation and refining, and/or chemical complexes. In another embodiment, the source of the feedstock is from an oil, gas, refinery, petrochemical operation, or any combination thereof. In an embodiment, the gas fermentation product is integrated into an infrastructure or process of an oil, gas, refinery, petrochemical operation, or any combination thereof, and the source of the feedstock is from an oil, gas, refinery, petrochemical operation, or any combination thereof.

In some embodiments, distillation may be employed to purify a product gas. In an embodiment, gas-liquid extraction may be employed. In an embodiment, a liquid product isolation may also be enriched via extraction using an organic phase. In another embodiment, purification may involve other standard techniques selected from ultrafiltration, one or more chromatographic techniques, or any combination thereof.

The method of the disclosure may further comprise separating a gas fermentation product from the fermentation broth. The gas fermentation product may be separated or purified from a fermentation broth using any method or combination of methods known in the art, including, for example, distillation, simulated moving bed processes, membrane treatment, evaporation, pervaporation, gas stripping, phase separation, ion exchange, or extractive fermentation, including for example, liquid-liquid extraction. As described in U.S. Pat. No. 2,769,321, the disclosure of which is incorporated by reference in its entirety herein, ethylene may be separated according to the method or combination of methods known in the art. In one embodiment, the ethylene produced is harvested from the bioreactor culture vessel.

In one embodiment, the gas fermentation product may be concentrated from the fermentation broth using reverse osmosis and/or pervaporation (U.S. Pat. No. 5,552,023). Water may be removed by distillation and the bottoms (containing a high proportion of gas fermentation product) may then be recovered using distillation or vacuum distillation to produce a high purity stream. Alternatively, with or without concentration by reverse osmosis and/or pervaporation, the gas fermentation product may be further purified by reactive distillation with an aldehyde (Atul, Chem Eng Sci, 59: 2881-2890, 2004) or azeotropic distillation using a hydrocarbon (U.S. Pat. No. 2,218,234). In another approach, the gas fermentation product may be trapped on an activated carbon or polymer absorbent from aqueous solution (with or without reverse osmosis and/or pervaporation) and recovered using a low boiling organic solvent (Chinn, Recovery of Glycols, Sugars, and Related Multiple —OH Compounds from Dilute-Aqueous Solution by Regenerable Adsorption onto Activated Carbons, University of California Berkeley, 1999). The gas fermentation product can then be recovered from the organic solvent by distillation. In certain embodiments, the gas fermentation product is recovered from the fermentation broth by continuously removing a portion of the broth from the bioreactor, separating microbial cells from the broth (conveniently by filtration), and recovering the gas fermentation product from the broth. Co-products, such as alcohols or acids may also be separated or purified from the broth. Alcohols may be recovered, for example, by distillation. Acids may be recovered, for example, by adsorption on activated charcoal. Separated microbial cells may be returned to the bioreactor in certain embodiments. Further, separated microbial cells may be recycled to the bioreactor in some embodiments. The cell-free permeate remaining after target products have been removed is also preferably returned to the bioreactor, in whole or in part. Additional nutrients (such as B vitamins) may be added to the cell-free permeate to replenish the medium before it is returned to the bioreactor.

Recovery of diols from aqueous media has been demonstrated a number of ways. Simulated moving bed (SMB) technology has been used to recover 2,3-butaendiol from an aqueous mixture of ethanol and associated oxygenates (U.S. Pat. No. 8,658,845). Reactive separation has also been demonstrated for effective diol recovery. In some embodiments, recovery of ethylene glycol is conducted by reaction of the diol-containing stream with aldehydes, fractionation and regeneration of the diol, final fractionation to recover a concentrated diol stream. See, e.g., U.S. Pat. No. 7,951,980.

In one embodiment, the method comprises recovering ethylene produced as disclosed above. In one embodiment, the method further comprises converting or using ethylene in the production of one or more chemical products following recovery of ethylene.

Ethylene is a high value gaseous compound which is widely used in industry. In an embodiment, ethylene may be used as an anaesthetic or as a fruit ripening agent, as well as in the production of a number of other chemical products. In some embodiments, ethylene may be used to produce polyethylene and other polymers, such as styrene, polystyrene, ethylene oxide, ethylene dichloride, ethylene dibromide, ethyl chloride and ethylbenzene. Ethylene oxide is, for example, a key raw material in the production of surfactants and detergents and in the production of ethylene glycol, which is used in the automotive industry as an antifreeze product. In one embodiment directed to ethylene dichloride, ethylene dibromide, and ethyl chloride may be used to produce products such as polyvinyl chloride, trichloroethylene, perchloroethylene, methyl chloroform, polyvinylidene chloride and copolymers, and ethyl bromide. In an embodiment, ethylbenzene is a precursor to styrene, which is used in the production of polystyrene (used as an insulation product) and styrene-butadiene (which is rubber suitable for use in tires and footwear). In another embodiment, a product is an ethylene propylene diene monomer (EPDM) rubber, an ethylene propylene (EPR/EPM) rubber, or any combination thereof.

It should be appreciated that the methods of the invention may be integrated or linked with one or more methods for the production of downstream chemical products from ethylene. In some embodiments, the methods of the invention may feed ethylene directly or indirectly to chemical processes or reactions sufficient for the conversion or production of other useful chemical products.

In some embodiments, ethylene is converted into hydrocarbon liquid fuels. In an embodiment, ethylene is oligomerized over a catalyst to selectively produce target products selected from gasoline, condensate, aromatics, heavy oil diluents, distillates, or any combination thereof. In other embodiments, the distillates are selected from diesel, jet fuel, sustainable aviation fuel (SAF), or any combination thereof.

In one embodiment, ethylene oligomerization is utilized towards desirable products. In an embodiment, oligomerization of ethylene may be catalyzed by a homogeneous catalyst, heterogeneous catalyst, or any combination thereof and having transition metals as active sites. In some embodiments, ethylene is further converted into long chain hydrocarbons by oligomerization. In other embodiments, straight chain olefins are the main product from ethylene oligomerization. In some embodiments, alpha olefins are the main product from ethylene oligomerization. In an embodiment, olefins are subjected to upgrading processes. In some embodiments, the upgrading process of olefins is hydrogenation. In an embodiment, olefins are subjected to olefin conversion technology. In some embodiments, the ethylene is incorporated in or converted to sustainable aviation fuel (SAF). In one embodiment, ethylene is interconverted to propylene, 2-butenes, or any combination thereof. In an embodiment, propylene is converted to polypropylene.

As a raw material, ethylene can used in the manufacture of polymers such as polyethylene (PE), polyethylene terephthalate (PET) and polyvinyl chloride (PVC) as well as fibres and other organic chemicals. These products are used in a wide variety of industrial and consumer markets such as the packaging, transportation, electrical/electronic, textile and construction industries as well as consumer chemicals, coatings and adhesives.

Ethylene can be chlorinated to ethylene dichloride (EDC) and can then be cracked to make vinyl chloride monomer (VCM). Nearly all VCM is used to make polyvinyl chloride which has its main applications in the construction industry.

Other ethylene derivatives include alpha olefins which are used in Linear low-density polyethylene (LLDPE) production, detergent alcohols and plasticizer alcohols; vinyl acetate monomer (VAM) which is used in adhesives, paints, paper coatings and barrier resins; and industrial ethanol which is used as a solvent or in the manufacture of chemical intermediates such as ethyl acetate and ethyl acrylate. Ethylene may be converted into ethylene-vinyl acetate (EVA), or poly(ethylene-vinyl acetate) (PEVA). EVA may be converted to thermoplastics materials. EVA may be incorporated in or used to make hot melt adhesives, hot glue sticks, soccer cleats, plastic wraps, craft foam sheets, and foam stickers. EVA may be incorporated in or used to make a drug delivery device. In some embodiments, EVA may be used to make foam. In one embodiment, EVA foam is used as padding in equipment for sports, including ski boots, bicycle saddles, hockey pads, boxing and mixed-martial-arts gloves and helmets, wakeboard boots, waterski boots, fishing rods and fishing-reel handles. In some embodiments EVA foam is used as a shock absorber in sports shoes. EVA may be used as EVA-based compression-moulded foam. EVA may be incorporated in or used to make floats for commercial fishing gear and floating eyewear. EVA can be incorporated or used to make encapsulation material for crystalline silicon solar cells. In some embodiments, EVA may be incorporated in or used to make slippers, sandals, fishing rods, substitute for cork, packaging, textile, bookbinding, bonding plastic films, metal surfaces, coated paper, redispersible powders in plasters and cement renders, and coating formulations in interior water-borne paints. EVA may undergo hydrolysis to provide ethylene vinyl alcohol (EVOH) copolymers. EVA may be used in orthotics, surfboard and skimboard traction pads, car mats, artificial flowers, a cold flow improver for diesel fuel, as a separator in HEPA filters, thermoplastic mouthguards, for conditioning and waterproofing leather, in nicotine transdermal patches, and plastic model kit parts.

Ethylene may further be used as a monomer base for the production of various polyethylene oligomers by way of coordination polymerization using metal chloride or metal oxide catalysts. The most common catalysts consist of titanium (III) chloride, the so-called Ziegler-Natta catalysts. Another common catalyst is the Phillips catalyst, prepared by depositing chromium (VI) oxide on silica.

Polyethylene oligomers so produced may be classified according to its density and branching. Further, mechanical properties depend significantly on variables such as the extent and type of branching, the crystal structure, and the molecular weight. There are several types of polyethylene which may be generated from ethylene, including, but not limited to:
Ultra-high-molecular-weight polyethylene (UHMWPE);
Ultra-low-molecular-weight polyethylene (ULMWPE or PE-WAX);
High-molecular-weight polyethylene (HMWPE);
High-density polyethylene (HDPE);
High-density cross-linked polyethylene (HDXLPE);
Cross-linked polyethylene (PEX or XLPE);
Medium-density polyethylene (MDPE);
Linear low-density polyethylene (LLDPE);
Low-density polyethylene (LDPE);
Very-low-density polyethylene (VLDPE); and
Chlorinated polyethylene (CPE).

Low density polyethylene (LDPE) and linear low-density polyethylene (LLDPE) mainly go into film applications such as food and non-food packaging, shrink and stretch film, and non-packaging uses. High density polyethylene (HDPE) is used primarily in blow molding and injection molding applications such as containers, drums, household goods, caps and pallets. HDPE can also be extruded into pipes for water, gas and irrigation, and film for refuse sacks, carrier bags and industrial lining.

According to one embodiment, the ethylene formed from the disclosure described above may be converted to ethylene oxide via direct oxidation according to the following formula:

C₂H₄+O₂→C₂H₄O

The ethylene oxide produced thereby is a key chemical intermediate in a number of commercially important processes including the manufacture of monoethylene glycol. Other EO derivatives include ethoxylates (for use in shampoo, kitchen cleaners, etc.), glycol ethers (solvents, fuels, etc.) and ethanolamines (surfactants, personal care products, etc.).

According to one embodiment of the disclosure, the ethylene oxide produced as described above may be used to produce commercial quantities of monoethylene glycol by way of the formula:

(CH₂CH₂)O+H₂O→HOCH₂CH₂OH

According to another embodiment, the claimed microorganism can be modified in order to directly produce monoethylene glycol. As described in WO 2019/126400, the disclosure of which is incorporated by reference in its entirety herein, the microorganism further comprises one or more of an enzymes capable of converting acetyl-CoA to pyruvate; an enzyme capable of converting pyruvate to oxaloacetate; an enzyme capable of converting pyruvate to malate; an enzyme capable of converting pyruvate to phosphoenolpyruvate; an enzyme capable of converting oxaloacetate to citryl-CoA; an enzyme capable of converting citryl-CoA to citrate; an enzyme capable of converting citrate to aconitate and aconitate to iso-citrate; an enzyme capable of converting phosphoenolpyruvate to oxaloacetate; an enzyme capable of converting phosphoenolpyruvate to 2-phospho-D-glycerate; an enzyme capable of converting 2-phospho-D-glycerate to 3-phospho-D-glycerate; an enzyme capable of converting 3-phospho-D-glycerate to 3-phosphonooxypyruvate; an enzyme capable of converting 3-phosphonooxypyruvate to 3-phospho-L-serine; an enzyme capable of converting 3-phospho-L-serine to serine; an enzyme capable of converting serine to glycine; an enzyme capable of converting 5,10-methylenetetrahydrofolate to glycine; an enzyme capable of converting serine to hydroxypyruvate; an enzyme capable of converting D-glycerate to hydroxypyruvate; an enzyme capable of converting malate to glyoxylate; an enzyme capable of converting glyoxylate to glycolate; an enzyme capable of converting hydroxypyruvate to glycolaldehyde; and/or an enzyme capable of converting glycolaldehyde to ethylene glycol.

In one embodiment, the microorganism comprises one or more of a heterologous enzyme capable of converting oxaloacetate to citrate; a heterologous enzyme capable of converting glycine to glyoxylate; a heterologous enzyme capable of converting iso-citrate to glyoxylate; a heterologous enzyme capable of converting glycolate to glycolaldehyde; or any combination thereof. In some embodiments, wherein the heterologous enzyme capable of converting oxaloacetate to citrate is a citrate [Si]-synthase [2.3.3.1], an ATP citrate synthase [2.3.3.8]; or a citrate (Re)-synthase [2.3.3.3]; the heterologous enzyme capable of converting glycine to glyoxylate is an alanine-glyoxylate transaminase [2.6.1.44], a serine-glyoxylate transaminase [2.6.1.45], a serine-pyruvate transaminase [2.6.1.51], a glycine-oxaloacetate transaminase [2.6.1.35], a glycine transaminase [2.6.1.4], a glycine dehydrogenase [1.4.1.10], an alanine dehydrogenase [1.4.1.1], or a glycine dehydrogenase [1.4.2.1]; the heterologous enzyme capable of converting iso-citrate to glyoxylate is an isocitrate lyase [4.1.3.1]; the heterologous enzyme capable of converting glycolate to glycolaldehyde is a glycolaldehyde dehydrogenase [1.2.1.21], a lactaldehyde dehydrogenase [1.2.1.22], a succinate-semialdehyde dehydrogenase [1.2.1.24], a 2,5-dioxovalerate dehydrogenase [1.2.1.26], an aldehyde dehydrogenase [1.2.1.3/4/5], a betaine-aldehyde dehydrogenase [1.2.1.8], or an aldehyde ferredoxin oxidoreductase [1.2.7.5]; or any combination thereof.

Monoethylene glycol produced according to either of the described methods may be used as a component of a variety of products including as a raw material to make polyester fibers for textile applications, including nonwovens, cover stock for diapers, building materials, construction materials, road-building fabrics, filters, fiberfill, felts, transportation upholstery, paper and tape reinforcement, tents, rope and cordage, sails, fish netting, seatbelts, laundry bags, synthetic artery replacements, carpets, rugs, apparel, sheets and pillowcases, towels, curtains, draperies, bed ticking, and blankets.

MEG may be used on its own as a liquid coolant, antifreeze, preservative, dehydrating agent, drilling fluid or any combination thereof. The MEG produced may also be used to produce secondary products such as polyester resins for use in insulation materials, polyester film, de-icing fluids, heat transfer fluids, automotive antifreeze and other liquid coolants, preservatives, dehydrating agents, drilling fluids, water-based adhesives, latex paints and asphalt emulsions, electrolytic capacitors, paper, and synthetic leather.

Importantly, the monoethylene glycol produced may be converted to the polyester resin polyethylene terephthalate (“PET”) according to one of two major processes. The first process comprises transesterification of the monoethylene glycol utilizing dimethyl terephthalate, according to the following two-step process:
First Step

C₆H₄(CO₂CH₃)₂+2 HOCH₂CH₂OH→C₆H₄(CO₂CH₂CH₂OH)₂+2 CH₃OH
Second Step

nC₆H₄(CO₂CH₂CH₂OH)₂→[(CO)C₆H₄(CO₂CH₂CH₂O)]ₙ+nHOCH₂CH₂OH

Alternatively, the monoethylene glycol can be the subject of an esterification reaction utilizing terephthalic acid according to the following reaction:

nC₆H₄(CO₂H)₂+nHOCH₂CH₂OH→[(CO)C₆H₄(CO₂CH₂CH₂O)]n+2nH₂O

The polyethylene terephthalate produced according to either the transesterification or esterification of monoethylene glycol has significant applicability to numerous packaging applications such as jars and, in particular, in the production of bottles, including plastic bottles. It can also be used in the production of high-strength textile fibers such as Dacron, as part of durable-press blends with other fibers such as rayon, wool, and cotton, for fiber fillings used in insulated clothing, furniture, and pillows, in artificial silk, as carpet fiber, automobile tire yarns, conveyor belts and drive belts, reinforcement for fire and garden hoses, seat belts, nonwoven fabrics for stabilizing drainage ditches, culverts, and railroad beds, and nonwovens for use as diaper topsheets, and disposable medical garments.

At a higher molecular weight, PET can be made into a high-strength plastic that can be shaped by all the common methods employed with other thermoplastics. Magnetic recording tape and photographic film are produced by extrusion of PET film. Molten PET can be blow-molded into transparent containers of high strength and rigidity that are also virtually impermeable to gas and liquid. In this form, PET has become widely used in bottles, especially plastic bottles, and in jars.

The disclosure provides compositions comprising ethylene glycol produced by the microorganisms and according to the methods described herein. For example, the composition comprising ethylene glycol may be an antifreeze, preservative, dehydrating agent, or drilling fluid.

The disclosure also provides polymers comprising ethylene glycol produced by the microorganisms and according to the methods described herein. Such polymers may be, for example, homopolymers such as polyethylene glycol or copolymers such as polyethylene terephthalate. Methods for the synthesis of these polymers are well-known in the art. See, e.g., Herzberger et al., Chem Rev., 116(4): 2170-2243 (2016) and Xiao et al., Ind Eng Chem Res. 54(22): 5862-5869 (2015).

The disclosure further provides polyethylene glycol conjugates. In some embodiments, polyethylene glycol (PEG) conjugates include PEG conjugated to a biopharmaceutical, proteins, antibodies, anticancer drugs, or any combination thereof. In other embodiments, the PEG conjugate is diethyl terephthalate (DET). In some embodiments, the PEG conjugate is dimethoxyethane.

The disclosure further provides compositions comprising polymers comprising ethylene glycol produced by the microorganisms and according to the methods described herein. For example, the composition may be a fiber, resin, film, or plastic.

In one embodiment, ethanol or ethyl alcohol produced according to the method of the disclosure may be used in numerous product applications, including antiseptic hand rubs (WO 2014/100851), therapeutic treatments for methylene glycol and methanol poisoning (WO 2006/088491), as a pharmaceutical solvent for applications such as pain medication (WO 2011/034887) and oral hygiene products (U.S. Pat. No. 6,811,769), as well as an antimicrobial preservative (U.S. Patent Application No. 2013/0230609), engine fuel (U.S. Pat. No. 1,128,549), rocket fuel (U.S. Pat. No. 3,020,708), plastics, fuel cells (U.S. Pat. No. 2,405,986), home fireplace fuels (U.S. Pat. No. 4,692,168), as an industrial chemical precursor (U.S. Pat. No. 3,102,875), cannabis solvent (WO 2015/073854), as a winterization extraction solvent (WO 2017/161387), as a paint masking product (WO 1992/008555), as a paint or tincture (U.S. Pat. No. 1,408,091), purification and extraction of DNA and RNA (WO 1997/010331), and as a cooling bath for various chemical reactions (U.S. Pat. No. 2,099,090). In addition to the foregoing, the ethanol generated by the disclosed method may be used in any other application for which ethanol might otherwise be applicable.

In an additional embodiment, isopropanol or isopropyl alcohol (IPA) produced according to the method may be used in numerous product applications, including either in isolation or as a feedstock for the production for more complex products. Isopropanol may also be used in solvents for cosmetics and personal care products, de-icers, paints and resins, food, inks, adhesives, and pharmaceuticals, including products such as medicinal tablets as well as disinfectants, sterilisers and skin creams.

The IPA produced may be used in the extraction and purification of natural products such as vegetable and animal oil and fats. Other applications include its use as a cleaning and drying agent in the manufacture of electronic parts and metals, and as an aerosol solvent in medical and veterinary products. It can also be used as a coolant in beer manufacture, a coupling agent, a polymerisation modifier, a de-icing agent and a preservative.

Alternatively, the IPA produced according to the method of the disclosure may be used to manufacture additional useful compounds, including plastics, derivative ketones such as methyl isobutyl ketone (MIBK), isopropylamines and isopropyl esters. Still further, the IPA may be converted to propylene according to the following formula:

CH₃CH₂CH₂OH→CH₃—CH═CH₂

The propylene produced may be used as a monomer base for the production of various polypropylene oligomers by way of chain-growth polymerization via either gas-phase or bulk reactor systems. The most common catalysts consist of titanium (III) chloride, the so-called Ziegler-Natta catalysts and metallocene catalysts.

Polypropylene oligomers so produced may be classified according to tacticity and can be formed into numerous products by either extrusion or molding of polypropylene pellets, including piping products, heat-resistant articles such as kettles and food containers, disposable bottles (including plastic bottles), clear bags, flooring such as rugs and mats, ropes, adhesive stickers, as well as foam polypropylene which can be used in building materials. Polypropylene may also be used for hydrophilic clothing and medical dressings.

In some embodiments ethylene is used to produce polyethylene: a common plastic used in a variety of consumer products such as plastic bags, plastic films, geomembranes, containers including bottles, etc.

In an embodiment ethylene is used to make ethylene glycol: a raw material in the manufacture of polyester fibers for clothes, upholstery, carpet, and pillows. In another embodiment ethylene used in the production of antifreeze in cooling and heating systems.

In another embodiment, ethylene is used in ethylene oxide: used to make other chemicals that are used in making products such as detergents, thickeners, solvents, plastics, and various organic chemicals. In some embodiments ethylene oxide is used as a sterilizing agent for medical equipment and a fumigating agent.

In some embodiments ethylene is used in vinyl acetate: used to make other chemicals that are used in paints, adhesives, paper coatings, and textiles.

In other embodiments, ethylene is used in ethylene dichloride: for the production of vinyl chloride, which is used to make polyvinyl chloride (PVC). PVC is used to make a variety of plastic and vinyl products including pipes, wire and cable coatings, and packaging materials.

In some embodiments, ethylene is used in aluminum alkyls: used as catalysts to increase the efficiency of making ethylene and other chemicals.

In some embodiments, ethylene is used in Ethylene Propylene Rubber (EPR): used in electrical insulation, roofing membrane, radiator hoses in vehicles, and waterproofing sheets.

In other embodiments, ethylene is used in agriculture: as a plant hormone and is used in agriculture to force the ripening of fruits.

In some embodiments, ethylene is used to make styrene: which is then used to make polystyrene. Polystyrene is used in various consumer products like disposable cutlery, CD and DVD cases, and insulation material.

In embodiments, ethylene is used to produce alpha olefins: used as co-monomers in the production of polyethylene, as well as in the production of detergents and lubricants.

In one embodiment, ethylene is used to produce butadiene. In some embodiments the butadiene is used in rubber tires.

In an embodiment, a method for the continuous production of ethylene, the process comprising: passing a gaseous substrate to a bioreactor containing a culture of a recombinant C1-fixing microorganism capable of producing ethylene in a culture medium such that the microorganism converts the gaseous substrate to ethylene; and recovering the ethylene from the bioreactor.

In other embodiments, converting the ethylene into a component used to manufacture tires. In an embodiment, the ethylene is converted into a component used in tire threads.

The method according to an embodiment, wherein the tires are end-of-life tires.

The method according to an embodiment, wherein the gaseous substrate is derived from a process comprising tires.

The method according to an embodiment, wherein the gaseous substrate is derived from a product circularity process or a sustainable chemical process.

The method according to an embodiment, further comprising converting the ethylene to a component used to manufacture new tires.

The method according to an embodiment, comprising resin components selected from ethylene and other olefins bonded to synthetic components selected from butadiene and isoprene to form hybrid polymers used to manufacture tires.

One embodiment is directed to a method for producing a polymer from a gaseous substrate comprising a first gas fermentation process produces at least one first product selected from butadiene, isoprene, conjugated dienes, or any combination thereof and a second gas fermentation process produces at least one second product selected from ethylene and olefins, or any combination thereof, and wherein the at least one first product and at least one second product are copolymerized to form a polymer.

The method according to an embodiment, wherein the first gas fermentation process and the second gas fermentation process are run in parallel.

The method according to an embodiment, wherein the first gas fermentation process and the second gas fermentation process are both run continuously.

The method according to an embodiment, comprising a first gas fermentation process produces rubber component and a second gas fermentation process produces a resin component, and wherein the rubber component and resin component are copolymerized to form a polymer.

The method according to an embodiment, wherein the rubber component and resin component are copolymerized by a suitable polymerization catalyst.

The method according to an embodiment, wherein the rubber component is selected from butadiene, isoprene, conjugated dienes, or any combination thereof.

The method according to an embodiment, wherein the resin component is selected from ethylene, olefins, or any combination thereof.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, further comprising converting the polymer into a tire.

One embodiment for the circular production of tires from a gaseous substrate is directed to a first gas fermentation process to produce at least one first product selected from butadiene, isoprene, conjugated dienes, or any combination thereof; and a second gas fermentation process to produce at least one second product selected from ethylene and olefins, or any combination thereof, wherein the at least one first product and at least one second product are copolymerized to form a polymer, and wherein the substrate is derived from a process comprising tires.

The method according to an embodiment, wherein the substrate is derived from a process comprising end-of-life tires.

One embodiment is directed to a method for the circular production of tires, the method comprising: 1) passing a gaseous substrate to a first bioreactor containing a culture of a recombinant C1-fixing microorganism capable of producing at least one first product selected from butadiene, isoprene, conjugated dienes, or any combination thereof in a culture medium such that the microorganism converts the gaseous substrate to the at least one first product; and recovering the at least one first product from the bioreactor; 2) passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor; 3) polymerizing the at least one first product with the at least one second product in the presence of a suitable polymerization catalyst to form a hybrid polymer; and 4) converting the hybrid polymer into a tire.

The method according to an embodiment, wherein the suitable polymerization catalyst further comprises another component contained in a general polymerization catalyst composition containing a metallocene complex.

The method according to an embodiment, wherein the metallocene complex is a complex compound having one or more cyclopentadienyl groups or derivative cyclopentadienyl groups bonded to a central metal.

The method according to an embodiment, wherein the central metal is selected from a lanthanoid element, scandium, yttrium, or any combination thereof.

The method according to an embodiment, wherein the central metal is selected from samarium (Sm), neodymium (Nd), praseodymium (Pr), gadolinium (Gd), cerium (Ce), holmium (Ho), scandium (Sc), and yttrium (Y).

The method according to an embodiment, wherein the first bioreactor and the second bioreactor are run in parallel.

The method according to an embodiment, wherein both the first bioreactor and the second bioreactor are continuously operated.

The method according to an embodiment, wherein the substrates are derived from a process comprising end-of-life tires.

The method according to an embodiment further comprising converting the isoprenoid into a product selected from synthetic rubber, block polymers containing styrene, thermoplastic rubbers, pressure-sensitive or thermosetting adhesives, butyl rubber, terpenes selected from citral, linalool, ionones, myrcene, L-menthol, N,N-diethylnerylamine, geraniol, nerolidols, flavours, fragrances, fuel additive, plastics, polyisoprene,

The method according to an embodiment further comprising converting the butadiene into a product selected from styrene-butadiene rubber, synthetic rubber, tires, component of tires, thermoplastic rubber, shoes, shoe soles, adhesives, sealants, asphalt, polymer modification components, nylon, ABS resins, chloroprene/neoprene rubber, nitrile rubber, plastics, acrylics, acrylonitrile-butadiene-styrene resins, and synthetic elastomers.

One embodiment is directed to a method for chemical recycling, the method comprising: a pyrolysis, gasification, and/or partial oxidation process; provided to a gas fermentation process; provided to a chemical product manufacturing process to produce a product comprising butadiene, isoprenoid, ethylene, polyethylene terephthalate (PET), or any combination thereof; provided to a synthetic rubber production process; provided to a tire manufacturing process; provided to a process of using tires; provided a process for the collecting and shredding of used tires; and provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a product comprising butadiene, isoprenoid, ethylene, polyethylene terephthalate (PET), or any combination thereof; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

One embodiment is directed to a method for chemical recycling, the method comprising: 1) a pyrolysis, gasification, and/or partial oxidation process; 2) provided to a gas fermentation process; 3) provided to a chemical product manufacturing process to produce a commodity product; 4) provided to a synthetic rubber production process; 5) provided to a tire manufacturing process; 6) provided to a process of using tires; 7) provided a process for the collecting and shredding of used tires; and 8) provided back to the pyrolysis, gasification, and/or partial oxidation process.

Another embodiment is directed to a method for chemical recycling, the method comprising: 1) a pyrolysis, gasification, and/or partial oxidation process producing an effluent stream; 2) passing the effluent stream to a gas fermentation process to produce a product; 3) passing the gas fermentation product to a chemical product manufacturing process to produce a commodity product; 4) passing the commodity product to a synthetic rubber production process to produce synthetic rubber; 5) passing the synthetic rubber product to a tire manufacturing process to produce a tire; 6) providing the tire to a process of using tires; 7) passing the used tires to a process for the collecting and shredding of used tires; and 8) recycling used tires back to the pyrolysis, gasification, and/or partial oxidation process.

One embodiment is directed to provides a method and a genetically engineered microorganism capable of producing ethylene from a gaseous substrate, the microorganism comprising a heterologous nucleic acid encoding an ethylene-forming enzyme (EFE).

In some aspects of the method disclosed herein, the microorganism is a recombinant C1-fixing microorganism capable of producing ethylene from a gaseous substrate comprising a nucleic acid encoding a group of exogenous enzymes comprising ethylene-forming enzyme (EFE).

In some aspects of the microorganism disclosed herein, the microorganism is directed to a recombinant C1-fixing microorganism capable of switching cellular burden in production of ethylene, the microorganism comprising a nucleic acid encoding a group of exogenous enzymes comprising ethylene-forming enzyme (EFE) and one or more inducible promoters.

The microorganism of an embodiment, further comprising a nucleic acid encoding a group of exogenous enzymes comprising alpha-ketoglutarate permease (AKGP), wherein the nucleic acid is operably linked to a promoter.

The microorganism of an embodiment, wherein the microorganism is selected from the group consisting of Cupriavidus necator and Ralstonia eutropha.

The microorganism of an embodiment, wherein the microorganism is Cupriavidus necator.

The microorganism of an embodiment, further comprising a nucleic acid encoding alpha-ketoglutarate permease, wherein the nucleic acid is codon optimized for expression in the microorganism.

The microorganism of an embodiment, wherein the one or more inducible promoters is selected from an H₂ inducible promoter, a phosphate limited inducible promoter, a nitrogen limited inducible promoter, or any combination thereof.

The microorganism of an embodiment, wherein the inducible promoter is a phosphate limited inducible promoter.

The microorganism of an embodiment, wherein phosphate concentration is about 0-0.5 mM.

The microorganism of an embodiment, wherein phosphate concentration is about 0.52 mM.

The microorganism of an embodiment, wherein the EFE is codon optimized for expression in the microorganism.

The microorganism of an embodiment, further comprising a disruptive mutation in one or more genes.

The microorganism of an embodiment, wherein ethylene is converted into a derivative material selected from polyethylene (PE), polyethylene terephthalate (PET), polyvinyl chloride (PVC), ethylene vinyl acetate (EVA), sustainable aviation fuel (SAF), or any combination thereof.

The microorganism of an embodiment, wherein the gaseous substrate comprises CO₂ and an energy source.

The microorganism of an embodiment, wherein the gaseous substrate comprises CO₂, and H₂, O₂, or both.

One embodiment is directed to a method for the continuous production of ethylene, the process comprising: passing a gaseous substrate to a bioreactor containing a culture of a recombinant C1-fixing microorganism according to claim 1, in a culture medium such that the microorganism converts the gaseous substrate to ethylene; and recovering the ethylene from the bioreactor.

One embodiment is directed to a method of culturing the microorganism according to an embodiment, comprising growing the microorganism in a medium comprising a gaseous substrate, wherein the gaseous substrate comprises CO₂.

The method of an embodiment, wherein the gaseous substrate comprises an industrial waste product or off-gas.

The method of an embodiment, further comprising an energy source.

The method of an embodiment, wherein the energy source is provided intermittently.

The method of an embodiment, wherein the energy source is H₂.

One embodiment is a directed to a method comprising growing the microorganism in a medium comprising a gaseous substrate, wherein the gaseous substrate comprises CO₂ and an energy source.

The method of an embodiment further comprises co-producing ethylene and microbial biomass.

The method of an embodiment, wherein switching the cellular burden comprises a step of limiting the intracellular oxygen concentration.

The method of an embodiment, wherein switching the cellular burden comprises a step of limiting dissolved oxygen concentration.

The method of an embodiment, wherein the dissolved oxygen concentration is at least of about 0.5% saturation (% sat.) to 1.0% sat. and at most of about 50% sat. to 60% sat.

The method of an embodiment, wherein the dissolved oxygen concentration is at least of about 0.5% sat. to 1.0% sat. and at most of about 60% sat. to 70% sat.

The method of an embodiment, wherein the dissolved oxygen concentration is at least of about 0.5% sat. to 1.0% sat. and at most of about 70% sat. to 80% sat.

The method of an embodiment, wherein the dissolved oxygen concentration is at least of about 0.5% sat. to 1.0% sat. and at most of about 80% sat. to 90% sat.

The method of an embodiment, wherein the dissolved oxygen concentration is at least of about 0.5% sat. to 1.0% sat. and at most of about 90% sat. to 100% sat.

The method of an embodiment, wherein the dissolved oxygen concentration is at least of about 0.01% sat. to 1.0% sat. and at most of about 50% sat. to 60% sat.

The method of an embodiment, wherein the dissolved oxygen concentration is at least of about 0.01% sat. to 1.0% sat. and at most of about 60% sat. to 70% sat.

The method of an embodiment, wherein the dissolved oxygen concentration is at least of about 0.01% sat. to 1.0% sat. and at most of about 70% sat. to 80% sat.

The method of an embodiment, wherein the dissolved oxygen concentration is at least of about 0.01% sat. to 1.0% sat. and at most of about 80% sat. to 90% sat.

The method of an embodiment, wherein the dissolved oxygen concentration is at least of about 0.01% sat. to 1.0% sat. and at most of about 90% sat. to 100% sat.

The method of an embodiment, wherein O₂ is fed into an inlet from about 4 vol. % to about 30 vol. %.

The method of an embodiment, wherein the O₂ is fed into an inlet from about 1 vol. % to about 50 vol. %.

The method of an embodiment, wherein switching the cellular burden comprises a step of limiting steady state phosphate concentration.

The method of an embodiment, wherein switching the cellular burden comprises a step of limiting steady state phosphate concentration of about 0 mM to about 0.50 mM.

The method of an embodiment, wherein switching the cellular burden comprises a step of limiting steady state phosphate concentration of about 0.05 mM to about 0.50 mM.

The method of an embodiment, wherein switching the cellular burden comprises a step of limiting steady state phosphate concentration of about 0.01 mM to about 0.60 mM.

The method of an embodiment, wherein switching the cellular burden comprises a step of limiting steady state phosphate concentration of about 0.01 mM to about 0.70 mM.

The method of an embodiment, wherein switching the cellular burden comprises a step of limiting steady state phosphate concentration of about 0.01 mM to about 0.80 mM.

The method of an embodiment, wherein switching the cellular burden comprises a step of limiting steady state phosphate concentration of about 0.01 mM to about 0.90 mM.

The method of an embodiment, wherein switching the cellular burden comprises a step of limiting steady state phosphate concentration of about 0.01 mM to about 1.0 mM.

The method of an embodiment, wherein switching the cellular burden comprises a step of limiting steady state phosphate concentration of about 0.52 mM.

The method of an embodiment, wherein the microbial biomass is suitable as animal feed.

The method of an embodiment, wherein the gaseous substrate further comprises H₂, O₂, or both.

In some aspects of the microorganism disclosed herein, the microorganism produces a commodity chemical product, microbial biomass, single cell protein (SCP), one or more intermediates, or any combination thereof.

In some aspects, the microbial biomass has a unit value. In one embodiment, the microbial biomass has a market value.

In some aspects of the microorganism disclosed herein, the microorganism is derived from a parental bacterium selected from the group consisting of Cupriavidus necator.

In some aspects of the microorganism disclosed herein, where the product is selected from the group 1-butanol, butyrate, butene, butadiene, methyl ethyl ketone, ethylene, acetone, isopropanol, lipids, 3-hydroxypropionate, terpenes, isoprene, fatty acids, fatty alcohols, 2-butanol, 1,2-propanediol, 1-propanol, 1-hexanol, 1-octanol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3-hexanediol, 3-methyl-2-butanol, 2-buten-1-ol, isovalerate, isoamyl alcohol, or monoethylene glycol.

The disclosure further provides the genetically engineered C1-fixing microorganism, further comprising a microbial biomass and at least one excipient.

The disclosure further provides the genetically engineered C1-fixing microorganism, wherein the animal feed is suitable for feeding to one or more of beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents.

The disclosure further provides the genetically engineered C1-fixing microorganism, wherein the microorganism is suitable as a single cell protein (SCP).

The disclosure further provides the genetically engineered C1-fixing microorganism, wherein the microorganism is suitable as a cell-free protein synthesis (CFPS) platform.

The disclosure further provides the genetically engineered C1-fixing microorganism, wherein the product is native to the microorganism.

In some aspects of the method disclosed herein, the substrate comprises one or more of CO, CO₂, and H₂.

According to another embodiment, the claimed microorganism can be modified in order to directly produce a commodity chemical as described in U.S. Patent Application Publication No. 2023/0092645A1, the disclosure of which is incorporated by reference herein. In one embodiment, wherein the commodity chemical is selected from ethanol, isopropanol, monoethylene glycol, sulfuric acid, propylene, sodium hydroxide, sodium carbonate, ammonia, benzene, acetic acid, ethylene oxide, formaldehyde, methanol, or any combination thereof. In one embodiment, the commodity chemical is aluminum sulfate, ammonia, ammonium nitrate, ammonium sulfate, carbon black, chlorine, diammonium phosphate, monoammonium phosphate, hydrochloric acid, hydrogen fluoride, hydrogen peroxide, nitric acid, oxygen, phosphoric acid, sodium silicate, titanium dioxide, or any combination thereof. In another embodiment, the commodity chemical is acetic acid, acetone, acrylic acid, acrylonitrile, adipic acid, benzene, butadiene, butanol, caprolactam, cumene, cyclohexane, dioctyl phthalate, ethylene glycol, methanol, octanol, phenol, phthalic anhydride, polypropylene, polystyrene, polyvinyl chloride, polypropylene glycol, propylene oxide, styrene, terephthalic acid, toluene, toluene diisocyanate, urea, vinyl chloride, xylenes, or any combination thereof. The method according to one embodiment, wherein the commodity chemical is utilized in the sector selected from plastics, synthetic fibers, synthetic rubber, dyes, pigments, paints, coatings, fertilizers, agricultural chemicals, pesticides, cosmetics, soaps, cleaning agent, detergents, pharmaceuticals, mining, or any combination thereof.

In another embodiment, the method includes incorporating a commodity chemical into one or more articles or converting a commodity chemical into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

In another embodiment, the method includes incorporating a commodity chemical into an article or converting a commodity chemical into a product selected from humectants, filters, fire extinguishing sprinkler system, fuel for warming foods, heat transfer fluids, non-reacted component in formulation, deodorizing or air purifying, softening agent, arts/craft glue/paste, toys, children products, freezer gel pack, treating wood rot and fungus, preserving biological tissues and organs, alkyd type resins, resin esters, enamels, lacquers, latex paint, asphalt emulsion, thermoplastic resin, hydrate inhibition agent, agent for removing water vapor, shoe polish, vaccines, screen cleaning solution, water-based hydraulic fluid, heat transfer for liquid cooled computers, personal lubricant, lubricant, toothpaste, anti-foaming agent in food industrial applications, flame resistant hydraulic fluids, additive for electrolytic polishing belts, industrial solvent, trash bags, shower curtains, cups, utensils, medical devices, durable goods, nondurable goods, plastic sacks, plastic lids, industrial strapping, construction materials, felt, ovenable trays, frozen food trays, microwavable tray, artificial vascular scaffolds, vascular prostheses, woven devices, polyester-based prostheses, vehicle liner material, soaps, cosmetic products, laundry detergent jugs, laundry detergent, soap microplastic, microbeads, cosmetic product microbeads, detergent pods, disinfectant with scrubbing agents, toothpaste with microbeads, face wash, conditioner, body wash, hand cleaner, exfoliating products, bath products, shower gels, powder laundry detergent, lotions, deodorants, toilet cleaners, sunscreen, shopping bags, mouthwash bottles, peanut butter containers, salad dressing and vegetable oil containers, polar fleece fiber, tote bags, paneling, milk jugs, juice bottles, bleach bottles, motor oil bottles, cereal box liners, recycling containers, floor tile, drainage pipe, benches, picnic tables, fencing, wire jacketing, sliding windows, decks, mud flaps, roadway gutters, speed bumps, squeezable bottles, bread, dry cleaning bags, trash can liners, trash cans, compost bins, shipping envelopes, lumber, syrup bottles, ketchup bottles, straws, medicine bottles, battery cables, battery cases, disposable plates and cups, egg cartons, carry-out containers, compact disc cases, signs and displays, synthetic fibers, yarn, stable phase change material, thermal energy storage material, nylon 6,6, nylon, tires, rubber, adiponitrile, shoes, footwear, or any combination thereof.

### EXAMPLES

The following examples further illustrate the disclosure but, of course, should not be construed to limit its scope in any way.

### Example 1: Ethylene Production from Formate as the Sole Carbon and Energy Source

The gene coding for ethylene forming enzyme was codon-adapted and synthesized for expression in Cupriavidus necator. The adapted gene along with constitutive promoter P10 were cloned into the broad host range expression vector pBBR1MCS2. The resulting products were used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 0.25 mL of culture was used to inoculate 25 mL of formate media in a 125 mL Erlenmeyer flask.

This culture was incubated for 48 hrs at 30° C. and 200 rpm with 65 mM formic acid added at varying times to control pH between 6.5-7.5. After 48 hrs, 20 mL of culture was transferred to a 160 mL serum bottle, 65 mM formic acid added, and the bottle sealed with an air-tight septa. Following an additional 16 hrs incubation, 60 mL of headspace volume was removed with an air-tight syringe and analyzed for ethylene production via GC. The sample was analyzed on a custom Wasson system for a variety of hydrocarbons and oxygenates. Ethylene was separated on a 50 m×0.53 um Wasson PN 2378 column and analyzed via GC FID.

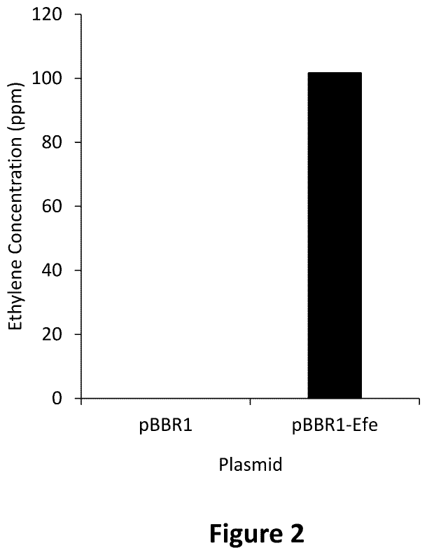

As shown in FIG. 2, the Cupriavidus necator strain with the Efe expressing plasmid (pBBR1-Efe) produced over 100 ppm ethylene while no ethylene was detected with the control strain containing the empty pBBR1 plasmid.

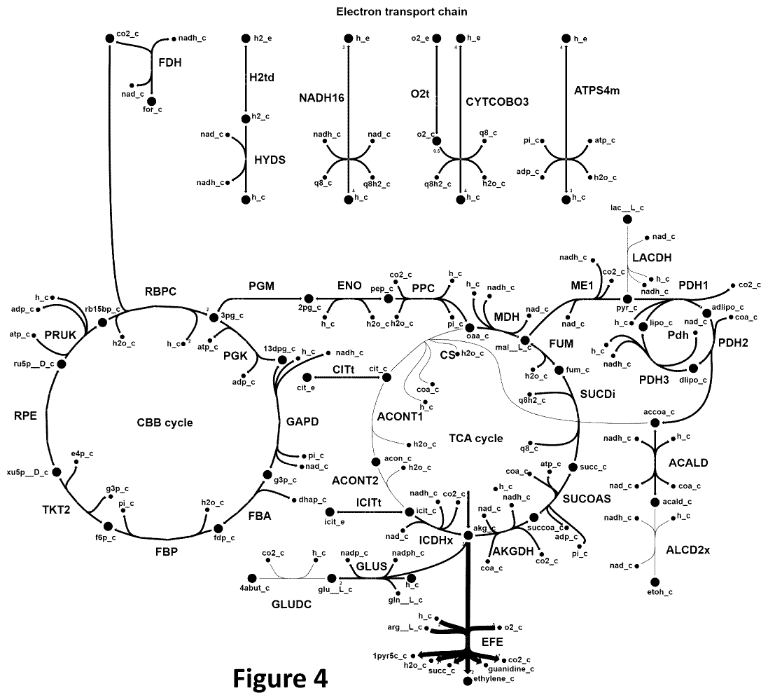

As shown in FIG. 4, the reaction abbreviations are the following: ACALD, Acetaldehyde dehydrogenase (acetylating); ACONT1, Aconitase (citrate hydro-lyase); ACONT2, Aconitase (isocitrate hydro-lyase); AKGDH, 2-Oxogluterate dehydrogenase; ALCD2x, Alcohol dehydrogenase (ethanol); ASPTA, Aspartate transaminase; ATPS4m, ATP synthase (four protons for one ATP); CITt, Citrate transport; CS, Citrate synthase; CYTCOBO3, Cytochrome oxidase bo3 (ubiquinol-8: 4 protons); EFE, ethylene-forming-reaction; ENO, Enolase; FBA, Fructose-bisphosphate aldolase; FBP, Fructose-bisphosphatase; FDH, Formate dehydrogenase; FUM, Fumarase; GAPD, Glyceraldehyde-3-phosphate dehydrogenase; GLUDC, Glutamate Decarboxylase; GLUS, Glutamate synthase; H₂td, Hydrogen transport; HYDS, Hydrogenase (NADH); ICDHx, Isocitrate dehydrogenase (NAD); ICITt, Isocitrate transport; LACDH, L-lactate dehydrogenase; MDH, Malate dehydrogenase; ME1, Malic enzyme (NAD); NADH16, NADH dehydrogenase (ubiquinone-8 and 3 protons); O2t, O2 transport (diffusion); PDH1, Pyruvate dehydrogenase E1 component; PDH2, Pyruvate dehydrogenase E2 component (dihydrolipoamide acetyltransferase); PDH3, Dihydrolipoamide dehydrogenase; PGK, Phosphoglycerate kinase; PGM, Phosphoglycerate mutase; PPC, Phosphoenolpyruvate carboxylase; PRUK, Phosphoribulokinase; RBPC, Ribulose-bisphosphate carboxylase; RPE, Ribulose 5-phosphate 3-epimerase; SUCDi, Succinate dehydrogenase (irreversible); SUCOAS, Succinyl-CoA synthetase (ADPforming); TKT2, Transketolase.

The genes listed are the following:
ACALD: H16_A1806 or H16_B0596 or H16_A2747 or H16_B0551
ACONT1: H16_A2638 or H16_B0568 or H16_A1907
ACONT2: H16_A2638 or H16_B0568 or H16_A1907
AKGDH: (H16_A2325 and H16_A2324 and H16_B1098) or (H16_A3724 and H16_A2325 and H16_A2324) or (H16_A2325 and H16_A2324 and H16_A1377) or (H16_A2323 and H16_A2325 and H16_A2324)
ALCD2x: H16_B2470 or H16_B0517 or H16_A3330 or H16_B1433 or H16_A0757 or H16_B1699 or H16_B1834 or H16_B1745
ASPTA: H16_A2857
ATPS4m: H16_A3643 and H16_A3642 and H16_A3639 and H16_A3636 and H16_A3637 and H16_A3638 and H16_A3640 and H16_A3641
CS: (H16_A2627 and H16_B0357 and H16_B2211) or (H16_A2627 and H16_B0357 and H16_A1229) or (H16_A2627 and H16_B0357 and H16_B0414)
CYTCOBO3: H16_A3396 and H16_A3397 and H16_A3398 and H16_A2319 and H16_A2318 and H16_A2316 and H16_B2062 and H16_B2059 and H16_A0342 and H16_A0343 and H16_A0347 and H16_A0345 ENO: H16_A1188
FBA: H16_B0278 or H16_B1384 or H16 A0568 or PHG416
FBP: H16_B1390 or H16_A0999 or PHG422
FDH: (H16_B1700 and H16_B1701) or (H16_A0640 and H16_A0642 and H16_A0641 and H16_A0644) or H16_A3292 or (H16_A2934 and H16_A2937 and H16_A2936 and H16_B1471) or (H16_B1454 and H16_B1452 and H16_B1453) or H16_B1383
FUM: H16_B0103 or H16 A2528
GAPD: H16_B1386 or H16_A3146 or PHG418
GLUDC: H16_A2930
GLUS: H16_B2194 or H16_A3430 or H16_B2192 or H16_A3431 or H16_B2193
HYDS: PHG088 and PHG089 and PHG090 and PHG091
ICDHx: H16_B1016
LACDH: H16_A0666
MDH: H16_B0334 or H16_A2634
ME1: H16_A3153
NADH16: H16_A1051 and H16_A1052 and H16_A1050 and H16_A1055 and H16_A1056 and H16_A1053 and H16_A1054 and H16_A1061 and H16_A1060 and H16_A1063 and H16_A1062 and H16_A1059 and H16_A1058 and H16_A1057 and H16_A0251
PDH1: H16_A1374 or H16_B1300 or H16_B0145 or H16_B2234 or H16_B2233 or H16_A1753
PDH2: H16_A1375 or H16_B0146
PDH3: H16_A3724 or H16_A2323 or H16_A1377 or H16_B1098
PGK: H16_A0566 or H16_B1385 or PHG417
PGM: H16_A0332 or H16_A0493
PPC: H16_A2921
PRUK: H16_B1389 or PHG421
RBPC: (PHG426 and PHG427) or (H16_B1394 and H16_B1395)
RPE: (H16_B1391 and H16_A3317) or (PHG423 and H16_A3317)
SUCDi: H16_B0204 and H16_A2632 and H16_A2631 and H16_A2630 and H16_A2629
SUCOAS: H16_A0548 and H16_A0547
TKT2: (H16_B1388 and H16_A3147) or (PHG420 and H16_A3147).

### Example 2: Continuous Ethylene Production from CO₂ with H₂ as the Energy Source

The gene coding for ethylene forming enzyme was codon-adapted and synthesized for expression in Cupriavidus necator. The adapted gene along with constitutive promoter P10 were cloned into the broad host range expression vector pBBR1MCS2. The resulting products were used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3−0.4 was reached.

100 mL of the above culture was used to inoculate a 1.4-L Infors HT Multifors 2 CSTR containing 600 mL of 2× startup media. The reactor was incubated at 30° C. and initiated with 250 rpm agitation and 150 nccm gas flow (3.14% O₂, 41% H₂, 3% CO₂, 52.86% N₂). Agitation and gas flow were ramped up to 1450 rpm and 750 nccm as the culture grew. When OD₆₀₀ exceeded 0.5, the culture was turned continuous using 4×_media with 7 μL/hr Pluronic 31R1 antifoam. The feed oxygen percentage was gradually increased to promote biomass production, with the balance taken off nitrogen percentage, subject to the constraint that the outlet oxygen percentage remain below 4.5% as a safety measure.

Gas samples from the reactor were plumbed via 305 stainless steel to a stream selection valve controlled by a microGC (manufacturer: Qmicro). Samples were then analyzed on a Rt-U BOND XP PLOT column under isothermal conditions (70° C.) via a thermal conductivity detector (TCD).

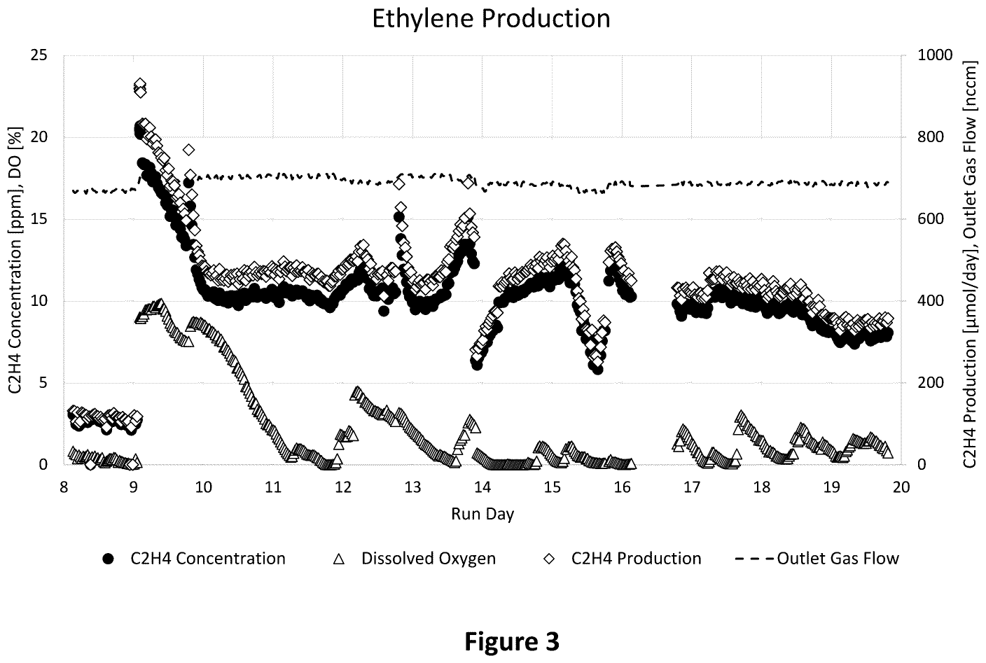

Once the culture was well-established, gas fractions were adjusted from O₂-limiting to H₂-limiting conditions such that a non-zero dissolved oxygen (DO) concentration was observed. Ethylene production varied as the system settled into steady-state and as gas fractions were adjusted, but production was maintained for over 11 days (FIG. 3). During this period, H₂ fraction ranged from 11-18% and O₂ fraction from 5.5-6.6%, with CO₂ held at 3% and N₂ as the balance. Upon switching back to O₂-limiting conditions, ethylene production ceased indicating the importance of oxygen availability for ethylene production.

### Example 3: Genome Scale Modeling of Gene Deletion Strategies to Eliminate Unwanted by-Products During Ethylene Production from CO₂ and H₂ in Cupriavidus necator

A genome-scale metabolic model of Cupriavidus necator like the one described by Park et al, BMC Systems Biology, 5: 101, 2011 was utilized to predict gene deletion(s) to eliminate unwanted by-products during ethylene production from CO₂ and H₂. The heterologous ethylene forming reaction was added to the wild type Cupriavidus necator model structure to represent the incorporation of the non-native compound production pathway. Ethylene production was simulated using constraint-based computational modeling techniques flux balance analysis (FBA) and linear minimization of metabolic adjustment (LMOMA) (Maia, Proceedings of the Genetic and Evolutionary Computation Conference Companion on—GECCO 'I7, New York, New York, ACM Press, 1661-1668, 2017) using cobrapy version 0.8.2 (Ebrahim., COBRApy: COnstraints-Based Reconstruction and Analysis for Python, BMC SystBiol, 7: 74, 2013), with optlang version 1.2.3 (Jensen, Optlang: An Algebraic Modeling Language for Mathematical Optimization,” The Journal of Open Source Software, 2, doi: 10.21105/joss.00139, 2017) as the solver interface and Gurobi Optimizer version 7.0.2 as the optimization solver.

TABLE 1
Gene deletions predicted to remove un-wanted by-products while
still allowing biomass formation and ethylene production:

| | Inactivated | |
|---|---|---|
| Eliminated | enzyme activity | Gene-reaction |
| by-product(s) | (reaction ID) | associations |
| Lactate | Lactate | H16_A0666 |
| | dehydrogenase | |
| | (LACDH) | |
| gamma- | Glutamate | H16_A2930 |
| aminobutyrate | decarboxylase | |
| | (GLUDC) | |
| citrate, | Citrate | (H16_A2627 and |
| iso-citrate | synthase (CS) | H16_B0357 and |
| | | H16_B2211) or |
| | | (H16_A2627 and |
| | | H16_B0357 and |
| | | H16_A1229) or |
| | | (H16_A2627 and |
| | | H16_B0357 and |
| | | H16_B0414) |
| Ethanol | Alcohol | H16_B2470 or H16_B0517 or |
| | dehydrogenase | H16_A3330 or H16_B1433 or |
| | (ALDC2x) | H16_A0757 or H16_B1699 or |
| | | H16_B1834 or H16_B1745 |
| Putrescine | Agmatinase | H16_A0044 |
| | (AGMT) | |
| iso-citrate | Aconitase 1 & 2 | H16_A2638 or H16_B0568 or |
| | (ACONT1, ACONT2) | H16_A1907 |
| Putrescine | Arginine | H16_A2930 |
| | decarboxylase | |
| | (ARGDC) | |
| Urocanate | Histidine | H16_A3018 |
| | ammonia-lyase | |
| | (HISAL) | |

### Example 4: Generating a Scarless Deletion at the Glutamate Decarboxylase (GLUDC) H16_A2930 Locus

Homology Arm Amplification and Assembly. To generate a scarless deletion of the glutamate decarboxylase (GLUDC) open reading frame in Cupriavidus necator H16, 500 bp homology arms were PCR amplified from C. necator H16 gDNA using Kappa 2× Master Mix and Gibson Assembled into the suicide plasmid pK18mobsacB using Thermo Fisher's GeneArt Seamless Cloning and Assembly Enzyme Mix. Briefly, the forward and reverse primers were designed in Geneious Prime to include 5′ and 3′ Gibson tails compatible with pK18mobsacB that was digested with the restriction enzyme BamHI. The primer and homology arm sequences are provided in Table ###below. Correct homology arm amplicon sizes were verified on a 1% agarose gel and column purified using a Zymo DNA Clean and Concentrator Kit. Purified homology arms were combined at a mass of 100 ng each with 100 ng of BamHI-digested pK18mobsacB in a 20 uL reaction with the GeneArt Seamless Cloning Enzyme Mix and incubated at room temperature for 30 mins. Next, 3 uL of the reaction mixture was used to transform chemically competent DH10B Escherichia coli and plated on LB agar medium containing 50 ng/uL kanamycin antibiotic. Plates were incubated for 24 h at 37° C., and colonies were screened by PCR to verify the presence of each homology arm insert. Positive colonies were grown overnight in 5 mL of LB broth supplemented with 50 ng/uL kanamycin, prepped using a Qiagen MINI Prep Kit, and plasmids were sequence verified using an Illumina MiSeq System.

Transformation via Conjugation. The sequence verified pK18mobsacB plasmid containing left and right 500 bp homology arms was electroporated into S17-1 E. coli cells and plated on LB supplemented with 50 ng/uL kanamycin. A single colony was picked and used to inoculate 5 mL of LB broth supplemented with 50 ng/uL kanamycin to generate the conjugation donor strain. To generate the conjugation recipient strain, a single colony of C. necator H16 grown on LB agar supplemented with 300 ng/uL gentamycin was picked and used to inoculate 5 mL of LB broth supplemented with 300 ng/uL gentamycin. The donor E. coli culture was grown overnight at 37° C. with shaking at 250 rpm, while the recipient C. necator culture was grown overnight at 30° C. with shaking at 200 rpm. The following morning, cells were collected by centrifugation for 3 min at 6000 rpm at 25° C., and pellets were resuspended in 50 uL of LB medium. The 50 uL of donor cells and recipient cells were mixed and spotted on a sterile hydrophilic filter on LB agar without antibiotics.

Selection and Counter-Selection. Plates were incubated overnight at 30° C., and cells were removed from the filter the following morning by folding it in half using sterile forceps and transferring to a tube containing 1 mL of LB. The cells were dislodged from the filter by vortexing, and a serial dilution was prepared (10⁰ to 10⁻³) and plated at 100 uL volumes on LB agar containing 300 ng/uL kanamycin. Plates were incubated for 5 days at 30° C. Next, colonies were replica-patched on LB agar plates containing 300 ng/uL kanamycin and LB agar plates containing 20% sucrose plus 300 ng/uL kanamycin. Colonies that grew on kanamycin but not sucrose plus kanamycin (primary integrants) were streak purified on LB agar plates containing 300 ng/uL kanamycin and subsequently cultured overnight at 30° C. in LB broth without antibiotics. The following morning, 100 uL volumes of serial diluted (10⁰ to 10⁻¹) culture was plated overnight at 30° C. on agar containing 20% sucrose (without antibiotics) to select for secondary recombinants. Sucrose-resistant colonies were patched on LB agar containing 20% sucrose (with and without 300 ng/uL kanamycin) to select for recombinants that were sucrose-resistant and antibiotic-sensitive. Finally, 12 SucR, KanS colonies were prepped for PCR and sequencing to verify deletion of the GLUDC H16_A2930 locus.

TABLE 2
Primer and Homology Arm Sequences for GLUDC H16_A2930 locus:

| SEQ | Sequence | |
|---|---|---|
| ID NO | Type | Sequence |
| 1 | F-Primer | 5′-TCGAGCTCGGTACCCGGGCAGGCGAGGCGCCG-3′ |
| | Left | |
| | Homology | |
| | Arm | |
| 2 | R-Primer | 5′-GGATGCCCGGCGCCAGTCCGCGTT-3′ |
| | Left | |
| | Homology | |
| | Arm | |
| 3 | F-Primer | 5′-GACTGGCGCCGGGCATCCTTGATGGAAAG-3′ |
| | Right | |
| | Homology | |
| | Arm | |
| 4 | F-Primer | 5′-TGCAGGTCGACTCTAGAGCGTTCGTGGATTTCAAGGGC-3′ |
| | Right | |
| | Homology | |
| | Arm | |
| 5 | Left | 5′CAGGCGAGGCGCCGCGTGTATTTGATTGATACCAACGTCATCAGCG |
| | Homology | AAACGCGCAAGCGCGAGCGCGCCAACCCCGGCGTGCGCGCGTTCTTCC |
| | Arm | GGCAGGCGGCGCGGGAAGGTGCCGCGCTCTACCTGTCGGCGCTGACCG |
| | (500 bp) | TGGGCGAGCTCCAGCGTGGCGTAGCGCTGATCCGCCATCGCGGCGATA |
| | | CGGCGCAGGCCGAGCTGCTGGAGCAATGGCTGGCGACCGTGCTGGAGG |
| | | ATTTTGGCCGGCTGGTGTTGCCGGTCGATGCCGACGTCGCCCAGGTCT |
| | | GGGGCCAGCTGCGCGCGCCGCGGCCTGAGCACGCGCTGGACAAGTTCA |
| | | TTGCCGCCACCGCGCTGATCCATGACCTGACCATTGTCACGCGCAATG |
| | | TTGAGGATTTCCGCGGCACGGGCGCGATGCTGCTGAATCCGTTCACCT |
| | | AGCCCCACCTCAAAAGAAAGGACCCCGCATAGCGGGGCCCTGCTCACG |
| | | TCAGCGGAACGCGGACTGGCGC-3′ |
| 6 | Right | 5′-CGGGCATCCTTGATGGAAAGCAGGACATAAAGGACTTGAGCCACA |
| | Homology | TGGCCGGCATGAGGTTCCCCGCTGCGGCGACATGTGGCTCTGACGTCA |
| | Arm | GGTGTGGCGGCCATTCTCGGGGCCAGCCTTCCGGCAAACGCCGGCATT |
| | (500 bp) | GTAATGCGCTCAGGTCTTCGGCAGTGTGACACCGTGCTGGCCCTGGTA |
| | | CTTGCCGCCGCGGTCGCGGTACGAGGTCTCGCAGACTTCGTCGCTCTC |
| | | GAAGAACAGCACCTGGGCGCAACCCTCGCCGGCGTAGATCTTGGCGGG |
| | | CAGCGGCGTGGTGTTGGAGAACTCCAGCGTCACATAGCCTTCCCACTC |
| | | CGGCTCGAACGGCGTCACATTGACGATGATGCCGCAGCGGGCGTAGGT |
| | | GCTCTTGCCCAGGCAGATGGTCAGCACGCTGCGCGGGATCCGGAAGTA |
| | | TTCCATCGTGCGCGCCAGCGCGAACGAATTGGGCGGGATGATGCAGAC |
| | | ATCGCCCTTGAAATCCACGAACG-3′ |
| 7 | pK18mob | 5′-TGCCGCAAGCACTCAGGGCGCAAGGGCTGCTAAAGGAAGCGGAAC |
| | sacB | ACGTAGAAAGCCAGTCCGCAGAAACGGTGCTGACCCCGGATGAATGTC |
| | empty | AGCTACTGGGCTATCTGGACAAGGGAAAACGCAAGCGCAAAGAGAAAG |
| | suicide | CAGGTAGCTTGCAGTGGGCTTACATGGCGATAGCTAGACTGGGCGGTT |
| | plasmid | TTATGGACAGCAAGCGAACCGGAATTGCCAGCTGGGGCGCCCTCTGGT |
| | | AAGGTTGGGAAGCCCTGCAAAGTAAACTGGATGGCTTTCTTGCCGCCA |
| | | AGGATCTGATGGCGCAGGGGATCAAGATCTGATCAAGAGACAGGATGA |
| | | GGATCGTTTCGCATGATTGAACAAGATGGATTGCACGCAGGTTCTCCG |
| | | GCCGCTTGGGTGGAGAGGCTATTCGGCTATGACTGGGCACAACAGACA |
| | | ATCGGCTGCTCTGATGCCGCCGTGTTCCGGCTGTCAGCGCAGGGGCGC |
| | | CCGGTTCTTTTTGTCAAGACCGACCTGTCCGGTGCCCTGAATGAACTC |
| | | CAAGACGAGGCAGCGCGGCTATCGTGGCTGGCCACGACGGGCGTTCCT |
| | | TGCGCAGCTGTGCTCGACGTTGTCACTGAAGCGGGAAGGGACTGGCTG |
| | | CTATTGGGCGAAGTGCCGGGGCAGGATCTCCTGTCATCTCACCTTGCT |
| | | CCTGCCGAGAAAGTATCCATCATGGCTGATGCAATGCGGCGGCTGCAT |
| | | ACGCTTGATCCGGCTACCTGCCCATTCGACCACCAAGCGAAACATCGC |
| | | ATCGAGCGAGCACGTACTCGGATGGAAGCCGGTCTTGTCGATCAGGAT |
| | | GATCTGGACGAAGAGCATCAGGGGCTCGCGCCAGCCGAACTGTTCGCC |
| | | AGGCTCAAGGCGCGGATGCCCGACGGCGAGGATCTCGTCGTGACCCAT |
| | | GGCGATGCCTGCTTGCCGAATATCATGGTGGAAAATGGCCGCTTTTCT |
| | | GGATTCATCGACTGTGGCCGGCTGGGTGTGGCGGACCGCTATCAGGAC |
| | | ATAGCGTTGGCTACCCGTGATATTGCTGAAGAGCTTGGCGGCGAATGG |
| | | GCTGACCGCTTCCTCGTGCTTTACGGTATCGCCGCTCCCGATTCGCAG |
| | | CGCATCGCCTTCTATCGCCTTCTTGACGAGTTCTTCTGAGCGGGACTC |
| | | TGGGGTTCGCTAGAGGATCGATCCTTTTTAACCCATCACATATACCTG |
| | | CCGTTCACTATTATTTAGTGAAATGAGATATTATGATATTTTCTGAAT |
| | | TGTGATTAAAAAGGCAACTTTATGCCCATGCAACAGAAACTATAAAAA |
| | | ATACAGAGAATGAAAAGAAACAGATAGATTTTTTAGTTCTTTAGGCCC |
| | | GTAGTCTGCAAATCCTTTTATGATTTTCTATCAAACAAAAGAGGAAAA |
| | | TAGACCAGTTGCAATCCAAACGAGAGTCTAATAGAATGAGGTCGAAAA |
| | | GTAAATCGCGCGGGTTTGTTACTGATAAAGCAGGCAAGACCTAAAATG |
| | | TGTAAAGGGCAAAGTGTATACTTTGGCGTCACCCCTTACATATTTTAG |
| | | GTCTTTTTTTATTGTGCGTAACTAACTTGCCATCTTCAAACAGGAGGG |
| | | CTGGAAGAAGCAGACCGCTAACACAGTACATAAAAAAGGAGACATGAA |
| | | CGATGAACATCAAAAAGTTTGCAAAACAAGCAACAGTATTAACCTTTA |
| | | CTACCGCACTGCTGGCAGGAGGCGCAACTCAAGCGTTTGCGAAAGAAA |
| | | CGAACCAAAAGCCATATAAGGAAACATACGGCATTTCCCATATTACAC |
| | | GCCATGATATGCTGCAAATCCCTGAACAGCAAAAAAATGAAAAATATC |
| | | AAGTTTCTGAATTTGATTCGTCCACAATTAAAAATATCTCTTCTGCAA |
| | | AAGGCCTGGACGTTTGGGACAGCTGGCCATTACAAAACGCTGACGGCA |
| | | CTGTCGCAAACTATCACGGCTACCACATCGTCTTTGCATTAGCCGGAG |
| | | ATCCTAAAAATGCGGATGACACATCGATTTACATGTTCTATCAAAAAG |
| | | TCGGCGAAACTTCTATTGACAGCTGGAAAAACGCTGGCCGCGTCTTTA |
| | | AAGACAGCGACAAATTCGATGCAAATGATTCTATCCTAAAAGACCAAA |
| | | CACAAGAATGGTCAGGTTCAGCCACATTTACATCTGACGGAAAAATCC |
| | | GTTTATTCTACACTGATTTCTCCGGTAAACATTACGGCAAACAAACAC |
| | | TGACAACTGCACAAGTTAACGTATCAGCATCAGACAGCTCTTTGAACA |
| | | TCAACGGTGTAGAGGATTATAAATCAATCTTTGACGGTGACGGAAAAA |
| | | CGTATCAAAATGTACAGCAGTTCATCGATGAAGGCAACTACAGCTCAG |
| | | GCGACAACCATACGCTGAGAGATCCTCACTACGTAGAAGATAAAGGCC |
| | | ACAAATACTTAGTATTTGAAGCAAACACTGGAACTGAAGATGGCTACC |
| | | AAGGCGAAGAATCTTTATTTAACAAAGCATACTATGGCAAAAGCACAT |
| | | CATTCTTCCGTCAAGAAAGTCAAAAACTTCTGCAAAGCGATAAAAAAC |
| | | GCACGGCTGAGTTAGCAAACGGCGCTCTCGGTATGATTGAGCTAAACG |
| | | ATGATTACACACTGAAAAAAGTGATGAAACCGCTGATTGCATCTAACA |
| | | CAGTAACAGATGAAATTGAACGCGCGAACGTCTTTAAAATGAACGGCA |
| | | AATGGTACCTGTTCACTGACTCCCGCGGATCAAAAATGACGATTGACG |
| | | GCATTACGTCTAACGATATTTACATGCTTGGTTATGTTTCTAATTCTT |
| | | TAACTGGCCCATACAAGCCGCTGAACAAAACTGGCCTTGTGTTAAAAA |
| | | TGGATCTTGATCCTAACGATGTAACCTTTACTTACTCACACTTCGCTG |
| | | TACCTCAAGCGAAAGGAAACAATGTCGTGATTACAAGCTATATGACAA |
| | | ACAGAGGATTCTACGCAGACAAACAATCAACGTTTGCGCCGAGCTTCC |
| | | TGCTGAACATCAAAGGCAAGAAAACATCTGTTGTCAAAGACAGCATCC |
| | | TTGAACAAGGACAATTAACAGTTAACAAATAAAAACGCAAAAGAAAAT |
| | | GCCGATGGGTACCGAGCGAAATGACCGACCAAGCGACGCCCAACCTGC |
| | | CATCACGAGATTTCGATTCCACCGCCGCCTTCTATGAAAGGTTGGGCT |
| | | TCGGAATCGTTTTCCGGGACGCCCTCGCGGACGTGCTCATAGTCCACG |
| | | ACGCCCGTGATTTTGTAGCCCTGGCCGACGGCCAGCAGGTAGGCCGAC |
| | | AGGCTCATGCCGGCCGCCGCCGCCTTTTCCTCAATCGCTCTTCGTTCG |
| | | TCTGGAAGGCAGTACACCTTGATAGGTGGGCTGCCCTTCCTGGTTGGC |
| | | TTGGTTTCATCAGCCATCCGCTTGCCCTCATCTGTTACGCCGGCGGTA |
| | | GCCGGCCAGCCTCGCAGAGCAGGATTCCCGTTGAGCACCGCCAGGTGC |
| | | GAATAAGGGACAGTGAAGAAGGAACACCCGCTCGCGGGTGGGCCTACT |
| | | TCACCTATCCTGCCCGGCTGACGCCGTTGGATACACCAAGGAAAGTCT |
| | | ACACGAACCCTTTGGCAAAATCCTGTATATCGTGCGAAAAAGGATGGA |
| | | TATACCGAAAAAATCGCTATAATGACCCCGAAGCAGGGTTATGCAGCG |
| | | GAAAAGCGCTGCTTCCCTGCTGTTTTGTGGAATATCTACCGACTGGAA |
| | | ACAGGCAAATGCAGGAAATTACTGAACTGAGGGGACAGGCGAGAGACG |
| | | ATGCCAAAGAGCTCCTGAAAATCTCGATAACTCAAAAAATACGCCCGG |
| | | TAGTGATCTTATTTCATTATGGTGAAAGTTGGAACCTCTTACGTGCCG |
| | | ATCAACGTCTCATTTTCGCCAAAAGTTGGCCCAGGGCTTCCCGGTATC |
| | | AACAGGGACACCAGGATTTATTTATTCTGCGAAGTGATCTTCCGTCAC |
| | | AGGTATTTATTCGGCGCAAAGTGCGTCGGGTGATGCTGCCAACTTACT |
| | | GATTTAGTGTATGATGGTGTTTTTGAGGTGCTCCAGTGGCTTCTGTTT |
| | | CTATCAGCTCCTGAAAATCTCGATAACTCAAAAAATACGCCCGGTAGT |
| | | GATCTTATTTCATTATGGTGAAAGTTGGAACCTCTTACGTGCCGATCA |
| | | ACGTCTCATTTTCGCCAAAAGTTGGCCCAGGGCTTCCCGGTATCAACA |
| | | GGGACACCAGGATTTATTTATTCTGCGAAGTGATCTTCCGTCACAGGT |
| | | ATTTATTCGGCGCAAAGTGCGTCGGGTGATGCTGCCAACTTACTGATT |
| | | TAGTGTATGATGGTGTTTTTGAGGTGCTCCAGTGGCTTCTGTTTCTAT |
| | | CAGGGCTGGATGATCCTCCAGCGCGGGGATCTCATGCTGGAGTTCTTC |
| | | GCCCACCCCAAAAGGATCTAGGTGAAGATCCTTTTTGATAATCTCATG |
| | | ACCAAAATCCCTTAACGTGAGTTTTCGTTCCACTGAGCGTCAGACCCC |
| | | GTAGAAAAGATCAAAGGATCTTCTTGAGATCCTTTTTTTCTGCGCGTA |
| | | ATCTGCTGCTTGCAAACAAAAAAACCACCGCTACCAGCGGTGGTTTGT |
| | | TTGCCGGATCAAGAGCTACCAACTCTTTTTCCGAAGGTAACTGGCTTC |
| | | AGCAGAGCGCAGATACCAAATACTGTTCTTCTAGTGTAGCCGTAGTTA |
| | | GGCCACCACTTCAAGAACTCTGTAGCACCGCCTACATACCTCGCTCTG |
| | | CTAATCCTGTTACCAGTGGCTGCTGCCAGTGGCGATAAGTCGTGTCTT |
| | | ACCGGGTTGGACTCAAGACGATAGTTACCGGATAAGGCGCAGCGGTCG |
| | | GGCTGAACGGGGGGTTCGTGCACACAGCCCAGCTTGGAGCGAACGACC |
| | | TACACCGAACTGAGATACCTACAGCGTGAGCTATGAGAAAGCGCCACG |
| | | CTTCCCGAAGGGAGAAAGGCGGACAGGTATCCGGTAAGCGGCAGGGTC |
| | | GGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTT |
| | | TTGTGATGCTCGTCAGGGGGGCGGAGCCTATGGAAAAACGCCAGCAAC |
| | | GCGGCCTTTTTACGGTTCCTGGCCTTTTGCTGGCCTTTTGCTCACATG |
| | | TTCTTTCCTGCGTTATCCCCTGATTCTGTGGATAACCGTATTACCGCC |
| | | TTTGAGTGAGCTGATACCGCTCGCCGCAGCCGAACGACCGAGCGCAGC |
| | | GAGTCAGTGAGCGAGGAAGCGGAAGAGCGCCCAATACGCAAACCGCCT |
| | | CTCCCCGCGCGTTGGCCGATTCATTAATGCAGCTGGCACGACAGGTTT |
| | | CCCGACTGGAAAGCGGGCAGTGAGCGCAACGCAATTAATGTGAGTTAG |
| | | CTCACTCATTAGGCACCCCAGGCTTTACACTTTATGCTTCCGGCTCGT |
| | | ATGTTGTGTGGAATTGTGAGCGGATAACAATTTCACACAGGAAACAGC |
| | | TATGACATGATTACGAATTCGAGCTCGGTACCCGGGGATCCTCTAGAG |
| | | TCGACCTGCAGGCATGCAAGCTTGGCACTGGCCGTCGTTTTACAACGT |
| | | CGTGACTGGGAAAACCCTGGCGTTACCCAACTTAATCGCCTTGCAGCA |
| | | CATCCCCCTTTCGCCAGCTGGCGTAATAGCGAAGAGGCCCGCACCGAT |
| | | CGCCCTTCCCAACAGTTGCGCAGCCTGAATGGCGAATGGCGATAAGCT |
| | | AGCTTCACGC-3′ |

TABLE 3
Efe sequences:

| SEQ | | Source | |
|---|---|---|---|
| ID NO | Enzyme | Organism | Sequence |
| 8 | Ethylene | Pseudomonas | MTNLQTFELPTEVTGCAADISLGRALIQAWQKDGIFQIKTD |
| | Forming | syringae | SEQDRKTQEAMAASKQFCKEPLTFKSSCVSDLTYSGYVAS |
| | Enzyme | | GEEVTAGKPDFPEIFTVCKDLSVGDQRVKAGWPCHGPVPW |
| | | | PNNTYQKSMKTFMEELGLAGERLLKLTALGFELPINTFTDL |
| | | | TRDGWHHMRVLRFPPQTSTLSRGIGAHTDYGLLVIAAQDD |
| | | | VGGLYIRPPVEGEKRNRNWLPGESSAGMFEHDEPWTFVTP |
| | | | TPGVWTVFPGDILQFMTGGQLLSTPHKVKLNTRERFACAY |
| | | | FHEPNFEASAYPLFEPSANERIHYGEHFTNMFMRCYPDRITT |
| | | | QRINKENRLAHLEDLKKYSDTRATGS |
| 9 | Ethylene | Ralstonia | MTGLTTFHLPERILHSEAHRQLGQDMVAAWRADGIFQIALS |
| | Forming | solanacearum | TPQQHTTDEAFAQSRRFFELDFETKRRHVSELTYSGYIASRE |
| | Enzyme | | EITAGEADYSEIFTICPDIGMDDVRVREGWPCHGPVPWPGT |
| | | | AYRDRMTDFTGMLGAFGERLLQLTALGLGLDDMETFTRLT |
| | | | RDGWHHMRVLRFPTVQSSENARGIGAHTDYGLLVIAAQD |
| | | | DVGGLYVRPPIAGERRNRNWLPSESTAGMFEHDDGWTFIK |
| | | | PEPAVLTVFPGDFLQFLTGGHLMSTPHKVRLNTRERFAMA |
| | | | YFHEPNFDAWVEPLKADADTDVAPIHYGTHFTNMFMRCY |
| | | | PKRITTRRIEEQGLLDRLPALGEVA |
| 10 | Ethylene | Microcoleus | MTHKYQEKIEVSNLQIFHLPESITGIQSDIDIARQMIQAWRR |
| | Forming | asticus | DGIFHVAVNKIQERKSERTFAASRRFFGMPLESKSQFISDLT |
| | Enzyme | | YSGYIASGEEVTAGESDYSEIFTVCKDVPLNDRRVQAQWPC |
| | | | HGPAPWPDEDYQQSMKAYMDELGSIGEKLLKLTALGLELD |
| | | | DINALTELTKDGWHHMRVLRFPALSQKSTRGIGAHTDYGL |
| | | | LVIAAQDDVGGLYIRPPVEGEKRNRNWLPTESMGGMYENE |
| | | | EPWILVKPVPSVLTVFPGDILQFLINGYLLSTPHKVRLNTRE |
| | | | RFAIAYFHEPNFEACVRPLFAPSSDEHIHYGSHFTNMFMRC |
| | | | YPDRITTRRIIDENRLSILGVLKNEGLRRLTTAKKAIELQR |
| 11 | Ethylene | Myxococcus | MIELETFQLPQSVSGREADIALGLTMVRAWRRDGIFQVRMS |
| | Forming | stipitatus | PAQAEKSQRAFELSRHFFRQSLETKARCVSDLTYSGYIASG |
| | Enzyme | | QELTASEADLSEVFTVCRDVPLTDPRVQSKWPCHGPGPWP |
| | | | DESWRQGMQAHAEELGSVGERLLRLIALGLGLDIDALTTL |
| | | | THDGWHHMRVLRFPARSPTTTRGIGAHTDYGLLVIAAQDD |
| | | | VGGLYVRPPVEGEKRPRNWLPHESSAGMYEHDEPWTYVK |
| | | | PVPGVLTVFPGDILQFLTRGYLLSTPHKVVLNTRERFALAY |
| | | | FHEPQFEACVRPLSAPTRDEYIHYGTHFTNMFMRSYPDRVT |
| | | | TQRILDESRLTTLSWLRQEAVLRTAPLEAVPLQRAAG |
| 12 | Ethylene | Nostoc sp. | MTDLQTFDLPKSITGSQSDIDLAHQMIQAWRTDGIFQVATN |
| | Forming | ATCC 43529 | AIQTRKTENAFEASKRFFRMPLDFKSQCISNLTYSGYIASGE |
| | Enzyme | | EITAGESDYSEIFTICKDVRLDDVRVQAQWPCHGSVPWPDN |
| | | | NYHQNMKAFMDELGIMGEKLLKLVALGLELDDIDALTKLT |
| | | | RDGWHHMRVLRFPALSEKSTRGIGAHTDYGLLVIAAQDDV |
| | | | GGLYVRPPVEGEKRNRNWLSDESSAGMYENDQPWTFVKP |
| | | | VPKVLTVFPGDILQFMTHNYLLSTPHKVRLNTRERFALAYF |
| | | | HEPNFQACVRPLFDSSNDDYIHYGTHFTNMFMRCYPYRITT |
| | | | RRILDEDRLSVLELLRNEALGGMLRPKYKTLVPSYL |
| 13 | Ethylene | Scytonema sp. | MTDLQTFHLPKSITGTQSDIDTAREIIQAWRTDGIFQVATNT |
| | Forming | NIES-4073 | IQDRKTESAFEASRRFFRMPMKFKSQCISDLNYGGYIASGEE |
| | Enzyme | | VTAGKSDYSEIYTICKDIPLNDARVQAQWPCHGPMPWPDQ |
| | | | EYHQSMKVFMDELGLIGEKLLKLTALGLGLDDINALTKLT |
| | | | RDGWHHMRVLRFPTLSQKSARGIGAHTDYGLLVIAAQDD |
| | | | VGGLYIRPPVEGEKRNRNWLSTESMAGMYENDDPWTFVK |
| | | | PVPSVLTVFPGDILQFLTNGYLLSTPHKVRLNTRERFALAYF |
| | | | HEPNFDACVRPLFDPSSDEHIHYGTHFTNMFMRCYADRITT |
| | | | RRIINEDRLSILARLENKTLGRLTTMKNAYALQR |

Following sequence confirmation of the GLUDC H16_A2930 locus deletion, the EFE expressing construct described in Example 1 was transformed into this strain via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs. Fermentations for ethylene production on formate or CO2/H2 were conducted as described in above example.

Additional gene deletions and chromosomal integrations beneficial to ethylene production and/or reducing by-product formation were conducted as described above.

### Example 5: A System for Generating Bubbles within a Vessel

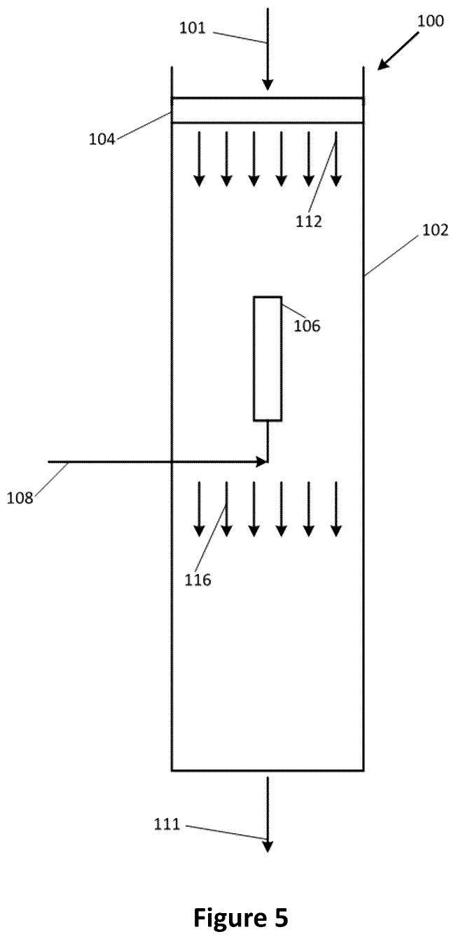

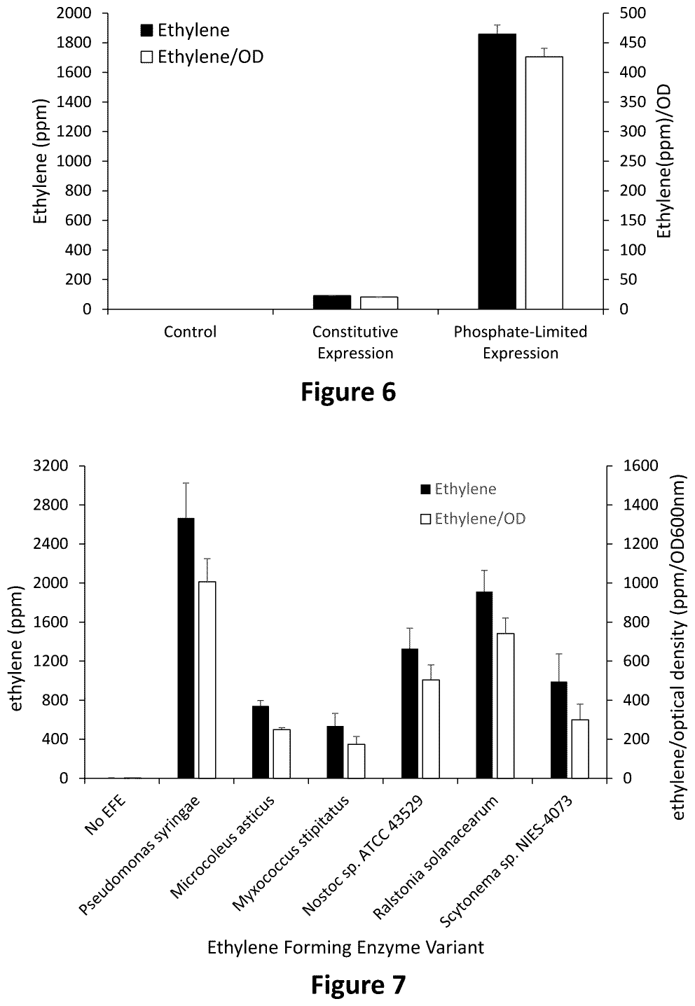

An example of a system of generating bubbles in a vessel 100 (FIG. 5). System 100 comprises cylindrical reactor 102. Liquid enters inlet or top portion 101 of reactor 102. The liquid may enter top portion 101 via an external pump in fluid communication with system 100. According to certain embodiments, the liquid entering top portion 101 is recirculated by an external pump in fluid communication with system 100. The liquid enters the top of perforated plate 104 and the liquid is accelerated by passing though the orifices in plate 104. According to certain examples, plate 104 may be configured to accelerate, for example, at least, greater than, less than, equal to, or any number from about 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99 to about 100% of the liquid in reactor 102. Sparger 106 injects gas bubbles into the liquid from gas source 108. Sparger 106 is positioned within reactor 102 such that a first zone is created in which the injected bubbles rise within reactor 102 and encounter accelerated liquid 112 exiting the bottom of plate 104. Accelerated liquid 112 from plate 104 breaks the rising bubbles into fine bubbles thereby increasing the superficial surface area required for the desired chemical or biological reaction. The fine bubbles may have a diameter in the range of about 0.1 mm to about 5 mm, or from about 0.5 mm to about 2 mm. In some examples, the fine bubbles may include a diameter from about 0.2 mm to 1.5 mm. According to another embodiment, the diameter of the fine bubbles may be, for example, at least, greater than, less than, equal to, or any number in between about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 to about 5.0 mm. Sparger 106 is further positioned within reactor 102 such that a second zone is created in which the fluid flow of liquid and fine bubbles may flow downward.

The fine bubbles may have a decreased rise velocity compared to the injected bubbles. Due to the overall flow of the accelerated liquid, fluid 116, containing the liquid and the fine bubbles, may have a net downward flow. The downward velocity of fluid 116 is greater than the overall rise velocity of the fine bubbles. Fluid 116 may exit reactor 102 at outlet 111. Plate 104 may have a thickness (and a depth of the orifices) from about 1 mm to 25 mm. According to another embodiment, the thickness of the plate may be, for example, at least, greater than, less than, equal to, or any number in between about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 mm.

The dimensions of the components of system 100, as illustrated in (FIG. 5), may vary depending upon the required use or process. According to certain embodiments, the diameter of the reactor 102 may be, for example, at least, greater than, less than, equal to, or any number in between about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5 to about 20.0 meters. According to other embodiments, the length of the reactor 102 may be, for example, at least, greater than, less than, equal to, or any number in between about 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.5, 21.5, 22.0, 22.5, 23.0, 23.5, 24.0, 24.5, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0 to about 50.0 meters.

The velocity of the liquid or a portion of the liquid accelerated from plate 104 can be determined by the following equation:

QL=N×(π/4)×d2×vj

where QL is the liquid volumetric flow rate (m3/s), vj is the jet velocity, N is the total number of orifices on the plate, d is the diameter of the orifices, and π is the mathematical symbol pi. According to one embodiment, the velocity of the accelerated liquid from plate 104 may be, for example, at least, greater than, less than, equal to, or any number in between about 5000, 5500, 6000, 6500, 7000, 7500, 8000, 8500, 9000, 9500, 10000, 10500, 11000, 11500, 12000, 12500, 13000, 13500, 14000, 14500, 15000, 15500, 16000, 16500, 17000, 17500, 18000, 18500, 19000, 19500 to about 20000 mm/s. As depicted in FIG. 5, the velocity of accelerated liquid 112 is critical to breaking bubbles injected into the liquid by sparger 106 into properly sized fine bubbles, and to ensuring that the fluid of liquid and fine bubbles has enough velocity to generate a net downward fluid flow. The superficial liquid velocity, VL, in the main reaction vessel may be calculated by the following equation: VL-QL/AC where QL is the volumetric flow rate of the liquid (m3/s) in the reaction vessel and AC is the cross-sectional area of the reaction vessel. Therefore, superficial liquid velocity represents velocity of the liquid phase if it occupied the entire cross-sectional area of the reaction vessel. According to embodiments, the superficial liquid velocity may also include zones or voids of stagnant liquid and fine bubbles, and/or net downward fluid flow. For the same liquid flow rate, the gas flow rate can vary depending on the actual application. Superficial velocity of the gas phase VG may be determined by the following equation: VG=QG/AC where QG is the volumetric flow rate of the gas (m3/s) injected into the liquid from the sparger(s) and AC is the cross-sectional area of the reaction vessel. According to another embodiment, the superficial velocity of the gas phase in the vessel may be, for example, at least, greater than, less than, equal to, or any number in between about 5, 10, 15, 20, 25, 30, 35, 40, 45, 50, 55, 60, 65, 70, 75, 80, 85, 90, 95 to about 100 mm/s. According to still another embodiment, the superficial velocity of the gas phase in the vessel may be, for example, approximately 50-60 mm/s.

Positioning of a sparger or multiple spargers 106 within reactor 102, and in an upper portion of reactor 102 has the additional advantage of decreasing hydrostatic pressure at the top of reactor 102 facilitating increased gas to liquid mass transfer rates with decreased energy requirements. Further, required reactor components are minimized, yet gas to liquid mass transfer rates are maximized with a smaller reactor footprint due to decreased reactor size. In some embodiments, for example, the systems and methods disclosed herein achieve gas to liquid mass transfer rates of at least 125 m³/min. In other examples, the gas to liquid mass transfer rates may be, for example, at least, greater than, less than, equal to, or any number in between about 100, 105, 110, 115, 120, 125, 130, 135, 140, 145, 150, 155, 160, 165, 170, 175, 180, 185, 190, 195 to about 200 m³/min. Additionally, the sparger configurations, superficial velocities of the gas and liquid phases achieved, and the increased gas to liquid mass transfer rates disclosed herein overcome known obstacles associated with the use of a gas and liquid phase system of the previous and conventional reactors. Particularly in bioreactors having a gas substrate and an aqueous culture.

### Example 6: Ethylene Production Using Diverse Genetic Sources of Ethylene-Forming Enzyme

Genes coding for ethylene forming enzyme from various organisms (accession numbers: Microcoleus asticus (NQE34890), Myxococcus stipitatus (WP_015351455.1), Nostoc sp. ATCC 43529 (RCJ18531), Ralstonia solanacearum (WP_014618742.1), Scytonema sp. NIES-4073 (WP_096562523.1) were cloned codon-adapted and synthesized for expression in Cupriavidus necator. The adapted gene along with a rhamnose inducible promoter (P_{rhaBAD}) and bicistronic RBS element were cloned into the broad host range expression vector pBBR1MCS2. The resulting products were used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 1 mL J minimal media with 10 g/L fructose, 10 g/L tryptone and 5 g/L yeast extract in a deep-well 96-well plate and grown for 24 hrs at 1000 rpm and 30° C. This pre-culture was used to inoculate (1%) 20 mL J minimal media with 10 g/L fructose in a 160 mL serum bottle. The cultures were then grown at 30° C. and 200 rpm for 6 hours, at which point 0.5 mM rhamnose was added. Following an additional 18 hrs of growth, the bottles were sealed with an air-tight septa. Following an additional 24 hrs incubation, 60 mL of headspace volume was removed with an air-tight syringe and analyzed for ethylene production via GC. The sample was analyzed on a custom Wasson system for a variety of hydrocarbons and oxygenates. Ethylene was separated on a 50m×0.53 um Wasson PN 2378 column and analyzed via GC FID.

As shown in FIG. 7, Cupriavidus necator strains with each of the Efe variants produced detectable ethylene, while no ethylene was detected using the control strain containing the empty pBBR1 plasmid. As these EFE variants range between 63-71% AA similarity to the canonical enzyme from Pseudomonas syringae, this demonstrates the ability for diverse EFE enzymes to enable ethylene production.

### Example 7: Use of Condition-Dependent Promoters for EFE Expression During Continuous Ethylene Production from CO₂ with H2 as the Energy Source

The gene coding for ethylene forming enzyme was codon-adapted and synthesized for expression in Cupriavidus necator. The adapted gene along with a phosphate-limited inducible promoter (Ppst-pho) and bicistronic RBS element were cloned into the broad host range expression vector pBBR1MCS2. The resulting products were used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

A single colony from a freshly streaked TSB plate was used to inoculate 3 mL TSB containing 50 mg/L chloramphenicol in a 14 mL Falcon round bottom polystyrene test tube with snap cap. Following overnight incubation at 30° C. and 200 rpm in a Thermo MAXQ shaker, 1 mL of culture was used to inoculate 100 mL LB in a 200 mL Schott bottle. Cells were grown at 30° C. and 200 rpm until an optical density of ˜0.3-0.4 was reached.

100 mL of the above culture was used to inoculate a 1.4-L Infors HT Multifors 2 CSTR under similar conditions to those described in Example 2.

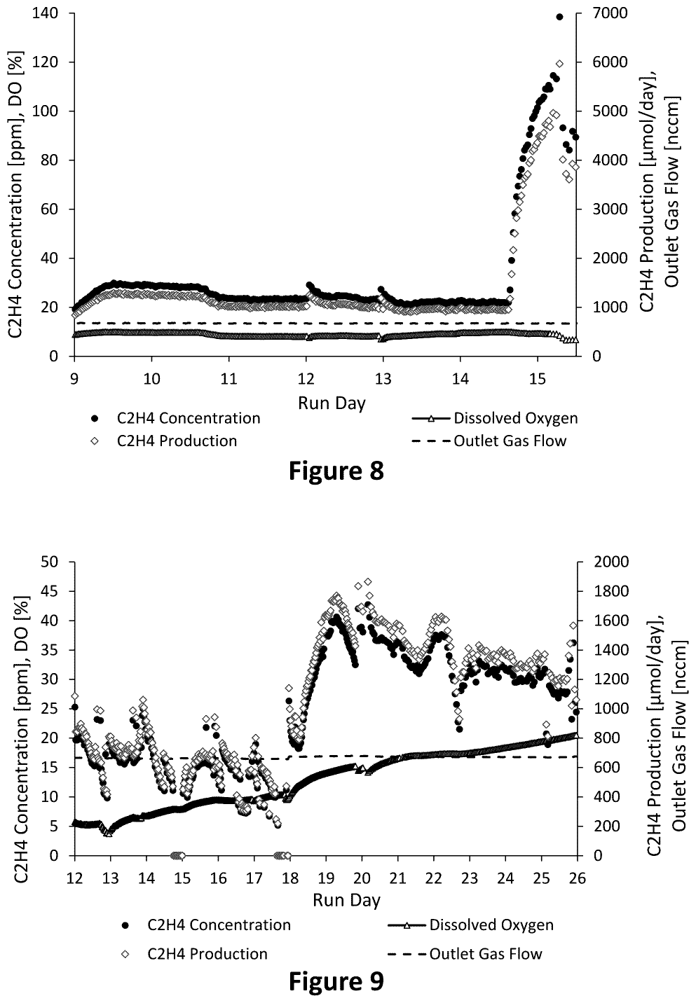

Once the culture was well-established, media compositions were adjusted to reach phosphate-limited conditions and further induce ethylene-forming enzyme expression (FIG. 8). This shift in media composition resulted in a ˜5-fold increase in ethylene concentration and production (FIG. 8) demonstrating the use of condition dependent promoters for ethylene production.

Similarly, the use of promoters responding to the gaseous carbon substrate, CO₂, and energy source, H₂, can also be used to express ethylene-forming enzyme to enable ethylene production in C. necator. Here, the adapted ethylene-forming enzyme gene and bicistronic RBS element were cloned into the broad host range expression vector pBBR1MCS2 with either the megaplasmid CbbL promoter (P_{cbbL,p}) responding to CO2 or the soluble hydrogenase promoter (P_{SH}) responding to H₂. The resulting products were used to transform E. coli and positive clones identified by PCR were confirmed by DNA sequencing. The sequence confirmed plasmid was then transformed into Cupriavidus necator PHB-4 via electroporation and selected on tryptic soy broth (TSB) agar plates containing 50 mg/L chloramphenicol. Transformants containing the pBBR1-Efe plasmid were confirmed via sequencing and a single colony then grown overnight in TSB at 30° C. and used to make glycerol stocks for storage at −80° C. Strain revival was conducted via streaking onto a TSB plate containing 50 mg/L chloramphenicol with incubation at 30° C. for 72 hrs.

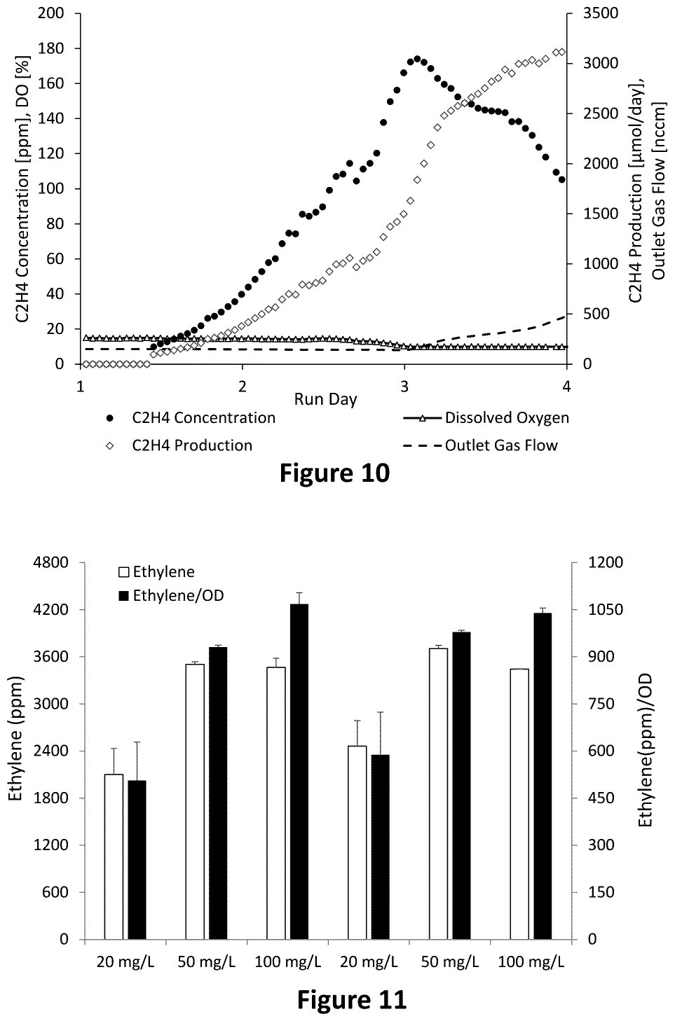

Following similar procedures to the above example (Example 2), these strains were run in a 1.4-L Infors HT Multifors 2 CSTR under similar conditions to those described in Example 2. Under these conditions, expression of ethylene-forming enzyme using the P_{CbbL,p} promoter resulted in the continuous production of ethylene from CO₂ and H2 for more than 2 weeks (FIG. 9). Furthermore, the use of a soluble hydrogenase promoter (P_{SH}) to drive ethylene-forming enzyme expression also enabled ethylene production, reaching concentrations over 100 ppm in the outlet gas stream (FIG. 10).

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Embodiments of the Disclosure

Embodiment 1. A recombinant C1-fixing microorganism capable of producing ethylene from a gaseous substrate comprising a nucleic acid encoding a group of exogenous enzymes comprising ethylene-forming enzyme (EFE).

Embodiment 2. A recombinant C1-fixing microorganism capable of switching cellular burden in production of ethylene, the microorganism comprising a nucleic acid encoding a group of exogenous enzymes comprising ethylene-forming enzyme (EFE) and one or more inducible promoters.

Embodiment 3. The microorganism according to embodiment 1, wherein the microorganism is selected from the group consisting of Cupriavidus necator and Ralstonia eutropha.

Embodiment 4. The microorganism according to embodiment 4, wherein the microorganism is Cupriavidus necator.

Embodiment 5. The microorganism according to embodiment 2, further comprising a nucleic acid encoding alpha-ketoglutarate permease, wherein the nucleic acid is codon optimized for expression in the microorganism.

Embodiment 6. The microorganism according to embodiment 2, wherein the one or more inducible promoters is selected from an H2 inducible promoter, a phosphate limited inducible promoter, a nitrogen limited inducible promoter, a CO2 inducible promoter, or any combination thereof.

Embodiment 7. The microorganism according to embodiment 2, wherein the EFE is codon optimized for expression in the microorganism.

Embodiment 8. The microorganism according to embodiment 1, further comprising a disruptive mutation in one or more genes.

Embodiment 9. The microorganism according to embodiment 1, wherein ethylene is converted into a derivative material selected from polyethylene (PE), polyethylene terephthalate (PET), polyvinyl chloride (PVC), ethylene-vinyl acetate (EVA), sustainable aviation fuel (SAF), or any combination thereof.

Embodiment 10. The microorganism according to embodiment 1, wherein the gaseous substrate comprises CO2 and an energy source.

Embodiment 11. The microorganism according to embodiment 1, wherein the gaseous substrate comprises CO2, and H2, O2, or both.

Embodiment 12. A method for the continuous production of ethylene, the process comprising: passing a gaseous substrate to a bioreactor containing a culture of a recombinant C1-fixing microorganism according to embodiment 1, in a culture medium such that the microorganism converts the gaseous substrate to ethylene; and recovering the ethylene from the bioreactor.

Embodiment 13. The method according to embodiment 12, wherein the gaseous substrate comprises an industrial waste product or off-gas.

Embodiment 14. The method according to embodiment 12, further comprising an energy source.

Embodiment 15. The method according to embodiment 12, wherein the energy source is provided intermittently.

Embodiment 16. The method according to embodiment 12, wherein the gaseous substrate comprises CO2 and an energy source.

Embodiment 17. The method according to embodiment 16, wherein the energy source is H2.

Embodiment 18. The method according to embodiment 16, wherein the gaseous substrate further comprises H2, O2, or both.

Embodiment 19. The method according to embodiment 12, further comprising a step of limiting dissolved oxygen concentration, thereby switching a cellular burden.

Embodiment 20. The method according to embodiment 12, further comprising controlling iron concentrations comprising at least 50 mg/L.

Embodiment 21. The method according to embodiment 12, further comprising converting the ethylene into a component used to manufacture tires.

Embodiment 22. The method according to embodiment 21, wherein the tires are end-of-life tires.

Embodiment 23. The method according to embodiment 12, wherein the gaseous substrate is derived from a process comprising tires.

Embodiment 24. The method according to embodiment 12, wherein the gaseous substrate is derived from a product circularity process or a sustainable chemical process.

Embodiment 25. The method according to embodiment 23, further comprising converting the ethylene to a component used to manufacture new tires.

1. A recombinant C1-fixing microorganism capable of producing ethylene from a gaseous substrate comprising a nucleic acid encoding a group of exogenous enzymes comprising ethylene-forming enzyme (EFE).

2. A recombinant C1-fixing microorganism capable of switching cellular burden in production of ethylene, the microorganism comprising a nucleic acid encoding a group of exogenous enzymes comprising ethylene-forming enzyme (EFE) and one or more inducible promoters.

3. The microorganism according to claim 1, wherein the microorganism is selected from the group consisting of Cupriavidus necator and Ralstonia eutropha.

4. The microorganism according to claim 3, wherein the microorganism is Cupriavidus necator.

5. The microorganism according to claim 2, further comprising a nucleic acid encoding alpha-ketoglutarate permease, wherein the nucleic acid is codon optimized for expression in the microorganism.

6. The microorganism according to claim 2, wherein the one or more inducible promoters is selected from an H₂ inducible promoter, a phosphate limited inducible promoter, a nitrogen limited inducible promoter, a CO₂ inducible promoter, or any combination thereof.

7. The microorganism according to claim 2, wherein the EFE is codon optimized for expression in the microorganism.

8. The microorganism according to claim 1, further comprising a disruptive mutation in one or more genes.

9. The microorganism according to claim 1, wherein ethylene is converted into a derivative material selected from polyethylene (PE), polyethylene terephthalate (PET), polyvinyl chloride (PVC), ethylene-vinyl acetate (EVA), sustainable aviation fuel (SAF), or any combination thereof.

10. The microorganism according to claim 1, wherein the gaseous substrate comprises CO₂ and an energy source.

11. The microorganism according to claim 1, wherein the gaseous substrate comprises CO₂, and H₂, O₂, or both.

12. A method for the continuous production of ethylene, the process comprising: passing a gaseous substrate to a bioreactor containing a culture of a recombinant C1-fixing microorganism according to claim 1, in a culture medium such that the microorganism converts the gaseous substrate to ethylene; and recovering the ethylene from the bioreactor.

13. The method according to claim 12, wherein the gaseous substrate comprises an industrial waste product or off-gas.

14. The method according to claim 12, further comprising an energy source.

15. The method according to claim 12, wherein the energy source is provided intermittently.

16. The method according to claim 12, wherein the gaseous substrate comprises CO₂ and an energy source.

17. The method according to claim 16, wherein the energy source is H₂.

18. The method according to claim 16, wherein the gaseous substrate further comprises H₂, O₂, or both.

19. The method according to claim 12, further comprising a step of limiting dissolved oxygen concentration, thereby switching a cellular burden.

20. The method according to claim 12, further comprising controlling iron concentrations comprising at least 50 mg/L.
